(19) United States
(12) Patent Application Publication
(10) Pub. No.: US 2026/0071956 A1
(43) Pub. Date: Mar. 12, 2026

(54) INFRARED-ACTIVATED RESONATOR AND WAVEGUIDE SYSTEM FOR MULTIMODAL TISSUE IMAGING AND BIOSENSING

(71) Applicant: MMSENSE Technologies Inc., Waterloo (CA)

(72) Inventors: Suren GIGOYAN, Waterloo (CA); Mohammad-Reza NEZHAD-AHMADI, Waterloo (CA)

(21) Appl. No.: 19/239,635

(22) Filed: Jun. 16, 2025

Related U.S. Application Data
(60) Provisional application No. 63/659,995, filed on Jun. 14, 2024.
(60) Provisional application No. 63/726,846, filed on Dec. 2, 2024.

(51) Int. Cl. G01N 21/359; G01N 21/47; G01N 33/569

(52) U.S. Cl. CPC G01N 21/359; G01N 21/47; G01N 33/56966

(57) ABSTRACT

Systems, devices, and methods for enhanced tissue imaging and biochemical detection using near-infrared (NIR) illumination in conjunction with resonant structures, such as whispering gallery mode (WGM) resonators and dielectric waveguides are proposed that operate in either transmission mode—where NIR light passes through a target sample—or reflection mode—where reflected NIR signals are recoupled into the sensing structure. These configurations support low-cost, flexible, real-time, and high-sensitivity analysis across a broad spectrum of biological and non-biological specimens, thereby enabling applications in tissue diagnostics, immunoassay-based detection, and material characterization, including deployment in point-of-care and resource-limited environments.

### CROSS-REFERENCE

This application is a non-provisional of, and claims all benefit, including priority, to U.S. Provisional Patent Application No. 63/659,095, filed on 14 Jun. 2024 and entitled “TISSUE IMAGING SYSTEM AND METHOD”, and U.S. Provisional Patent Application No. 63/726,846, filed on 2 Dec. 2024 and also entitled “TISSUE IMAGING SYSTEM AND METHOD” The entire content of the aforementioned provisional applications are hereby incorporated by reference in their entirety.

### FILED

Embodiments of the present disclosure relate to the field of imaging and biosensing technologies. More specifically, embodiments relate to systems, devices, and methods for enhanced tissue imaging and biochemical detection using near-infrared (NIR) illumination in conjunction with resonant structures, such as whispering gallery mode (WGM) resonators and dielectric waveguides.

### INTRODUCTION

Human tissue imaging and biochemical sensing are components of diagnostic pathology, biomedical research, and clinical decision-making.

Imaging modalities—such as X-ray mammography, computed tomography (CT), and ultrasound—have demonstrated clinical utility but are constrained by several limitations, including exposure to ionizing radiation, limited spatial resolution, suboptimal contrast differentiation, and procedural invasiveness.

Similarly, biochemical sensing techniques—such as fluorescence-based assays and colorimetric chemical methods—often require complex sample preparation, molecular labeling, and extended processing times, limiting their practicality for real-time or point-of-care use. In addition, many existing diagnostic systems are cost-prohibitive or lack portability. Accordingly, there is a recognized need for diagnostic platforms that are non-invasive, label-free, rapid, cost-effective, and capable of delivering high-resolution imaging and sensitive molecular detection across both centralized clinical settings and decentralized or resource-constrained environments.

Accordingly, there exists a need in the art for advanced diagnostic imaging and biosensing systems that address the limitations of existing modalities. Such systems should ideally be non-invasive, eliminate reliance on ionizing radiation, and provide enhanced spatial resolution and biochemical sensitivity. Further desirable characteristics include simplified system architecture, rapid response suitable for real-time analysis, low manufacturing and operational costs, and adaptability to compact, portable, or point-of-care configurations.

### SUMMARY

The systems and methods disclosed herein are directed toward addressing challenges in biomedical imaging and molecular diagnostics. A versatile imaging and biosensing system is disclosed, capable of performing specimen-agnostic diagnostic tasks, including high-resolution imaging of biological tissues and detection of target analytes in immunoassay configurations. A resonator works in combination with a receiver circuit, which is configured to identify certain characteristic changes in the resonator as a signal (e.g., infrared) travels through a tissue under analysis. The receiver circuit can include a network analyzer circuit, but this can be replaced with alternate electronic circuits during practical usage.

A physical imaging system is controlled by a computer processor operating in conjunction with physical sensors, the processor configured to support a transmission mode, a reflection mode, or both modes. In the transmission mode, near-infrared (NIR) radiation passes through a target sample—and in the reflection mode, NIR radiation is reflected from the sample and re-coupled into the sensing structure. Single mode and dual mode operation is contemplated in different proposed embodiments.

The system architecture incorporates resonant structures such as whispering gallery mode (WGM) resonators and dielectric waveguides, each designed to operate at high quality factors (Q-factors) to enhance signal sensitivity, spatial resolution, and detection accuracy. The resonator or waveguide interacts with a receiver circuit configured to detect characteristic variations in the electromagnetic response resulting from changes in sample properties under NIR illumination. In certain embodiments, the sample may comprise biological tissue or a biochemical assay substrate, such as a lateral flow assay (LFA) membrane.

Variations in scattering parameters, including S21, S11, S12, and S22, are monitored and analyzed to extract diagnostic information. The receiver circuitry may include a vector network analyzer (VNA) or alternative cost-efficient, compact electronic components adapted for real-time, portable, and field-deployable biosensing and imaging applications.

The proposed system may be implemented in a range of practical applications, including but not limited to the detection and monitoring of pathogens, illnesses, and disease biomarkers. Additional use cases encompass immunoassay analysis, tissue classification, and molecular diagnostics. Although various embodiments described herein focus on human tissue imaging for illustrative purposes, the disclosed systems and methods are broadly applicable to specimen-agnostic use scenarios. In particular, the platform is adaptable for both biological and non-biological imaging tasks, including non-destructive testing, materials analysis, and quality control in industrial settings. These and other advantages of the proposed approach are discussed in further detail throughout the present disclosure.

According to one aspect of the present disclosure, there is provided a tissue imaging system comprising: a receptacle configured to receive a tissue under test (TUT) sample; a resonant structure, such as a whispering gallery mode (WGM) resonator or dielectric waveguide; and an emitter configured to direct near-infrared (NIR) radiation through the TUT or biological sample and onto the resonant or waveguide structure.

The system is configured to detect variations in absorption or scattering characteristics at the resonator resulting from the interaction of the NIR radiation with the TUT sample. These variations are processed and converted into output signals indicative of one or more material properties of the TUT sample. In various embodiments, the tissue may include biological components such as adipose tissue, muscle, bone, or pathological formations, and different electromagnetic responses—such as shifts in resonance frequencies or changes in transmission/reflection coefficients—may be correlated with corresponding diagnostic parameters.

In one embodiment, the resonator comprises a whispering gallery mode (WGM) resonator formed from high-resistivity silicon. In other embodiments, the resonator may be constructed from alternative near-infrared (NIR) sensitive materials, including but not limited to germanium, gallium arsenide, or chalcogenide glass, or hybrid of magnetic material such as Ferrite combined with any of the NIR sensitive material selected based on their optical absorption characteristics, dielectric properties, and compatibility with the intended sensing application and required functionalities for both reciprocal and non-reciprocal sensing mechanism

In one embodiment, the near-infrared (NIR) radiation emitted by the emitter is directed to illuminate both a whispering gallery mode (WGM) resonator and a nearby coupling structure, which may include a microstrip transmission line or, in alternative embodiments, a dielectric waveguide. In further embodiments, the resonant sensing structure itself may be implemented as a NIR-sensitive dielectric waveguide, wherein optical illumination modulates the electromagnetic propagation characteristics of the waveguide. These changes may be monitored via transmission or reflection coefficients, enabling the detection of sample-induced perturbations in either resonator- or waveguide-based configurations.

In one embodiment, the whispering gallery mode (WGM) silicon-based resonator is doped with aluminum to modify its electrical conductivity and optical absorption properties under near-infrared (NIR) illumination. The doping facilitates enhanced photoconductive response, thereby improving the sensitivity of the resonator to variations in optical intensity and enabling more precise detection of sample-induced effects.

In one embodiment, variations in the optical absorption characteristics of the sample—caused by interaction with near-infrared (NIR) illumination—induce changes in the electromagnetic response of the resonant structure. These perturbations are manifested as variations in one or more scattering parameters, including but not limited to the magnitude and/or phase of the transmission coefficient (S21), reflection coefficient (S11), reverse transmission (S12), or reverse reflection (S22). In immunoassay-based implementations, such changes may correspond to specific biomolecular binding events that modulate light absorption and subsequently alter the resonator's dielectric properties, enabling label-free and real-time detection of target analytes.

In one embodiment, the system further comprises a translation stage configured to support the tissue under test (TUT) sample. The translation stage is operable to displace the TUT sample relative to the emitter and the resonant sensing structure along one or more spatial axes, thereby enabling spatial scanning for two-dimensional or three-dimensional imaging. In an alternative embodiment, spatial scanning may be achieved without mechanical displacement by employing a two-dimensional array of resonators or dielectric waveguides, each coupled to a corresponding emitter and/or detector. This array-based configuration enables real-time image acquisition across the scanned field, improving imaging speed and eliminating the need for mechanical motion.

By moving the sample, the tissue displacement allows distinguishing of different tissue types at different measurement points, and the data can be stored and collated into a two dimensional data structure. This configuration provides a high-contrast imaging modality capable of distinguishing between different tissue types, such as muscle and fat, with greater precision and safety compared to traditional X-ray-based techniques, where there are serious technical limitations relating to invasiveness and resolution, especially, but not limited to, relating to breast and brain imaging. The configuration can also be used to assess different tissue sub-types, such as distinguishing between normal tissue and abnormal tissue.

In one embodiment, the translation stage is configured to displace the tissue under test (TUT) sample along a two-dimensional plane defined by orthogonal X and Y axes. This planar motion enables raster-scanning of the sample relative to the emitter and resonant sensing structure, facilitating spatially resolved data acquisition. In certain embodiments, the translation stage is motorized and programmable, allowing for automated scanning sequences. In further embodiments, the stage may also be configured to enable displacement along a Z-axis, thereby supporting three-dimensional volumetric imaging or depth profiling of the sample.

In one embodiment, the system is configured to execute a pre-operational diagnostic or calibration procedure prior to the introduction of the tissue under test (TUT) sample into the receptacle. During this procedure, the emitter is operable to emit near-infrared (NIR) radiation at varying intensity levels directed toward the resonator or dielectric waveguide structure. The system monitors corresponding changes in electromagnetic response—such as resonance frequency shifts, Q-factor changes, or variations in scattering parameters (e.g., S21, S11, S12, S22)—to assess baseline operational characteristics. In configurations employing reflection-mode sensing, the system may evaluate the reflected NIR signal recoupled into the resonator to verify alignment and sensitivity. In certain embodiments, the system further comprises control logic or firmware capable of performing automated self-correction or compensation for drift, misalignment, or environmental variations based on the diagnostic data.

In one embodiment, the system further comprises a vector network analyzer (VNA) operatively coupled to the resonator or dielectric waveguide structure. The VNA is configured to perform frequency-domain measurements by sweeping across a designated frequency range and capturing scattering parameters—such as S21, S11, S12, and S22—associated with the resonator's response to incident near-infrared (NIR) illumination. Variations in the magnitude and/or phase of these parameters are analyzed to identify changes in absorption rates caused by the tissue under test (TUT) sample, and are converted into output signals indicative of the sample's dielectric, optical, or biochemical properties. In alternative embodiments, the VNA may be replaced with a compact signal processing module comprising analog or digital electronics, including direct conversion receivers or I/Q demodulation circuits. These modules may be integrated into embedded systems or application-specific integrated circuits (ASICs) to support miniaturized, low-cost, and field-deployable diagnostic platforms.

In one embodiment, the material properties derived from the tissue under test (TUT) sample include biochemical binding events, such as specific antigen-antibody interactions. These interactions may occur within or on a substrate in proximity to the resonator or dielectric waveguide, such as in an immobilized immunoassay layer or lateral flow membrane. The presence and degree of binding influence the local optical or electromagnetic properties of the system—such as effective permittivity, conductivity, or light absorption—which in turn modulate the resonator's response. These modulations are detected and quantified through changes in scattering parameters, enabling label-free biosensing of target biomolecules.

In one embodiment, the system is configured to operate in a reflection-mode biosensing configuration, wherein near-infrared (NIR) radiation is directed from an emitter toward a TUT sample or immunoassay membrane positioned adjacent to the resonator or dielectric waveguide. The reflected NIR signal, modulated by the sample's optical and biochemical characteristics, is re-coupled into the sensing structure. Specific antigen-antibody interactions occurring at or near the sample surface alter the intensity, phase, and spectral characteristics of the reflected signal. These interactions induce perturbations in the resonance behavior of the sensing structure, which are measured as changes in scattering parameters—such as S11 or S12—as well as shifts in resonance frequency. The combination of amplitude, phase, and frequency shift data provides enhanced specificity and sensitivity for detecting the presence and concentration of target biomolecules. In certain embodiments, the immunoassay is supported within a transparent or semi-transparent cartridge or microfluidic container that is optically aligned with the emitter and the resonant structure to ensure consistent and reproducible measurements.

According to the present disclosure, there is further provided a method for operating a tissue imaging system, comprising receiving, at a receptacle, a tissue under test (TUT) sample to be imaged, the receptacle positioning the TUT sample between a resonator and an emitter, directing, at the emitter, near infrared light through the TUT sample and onto the resonator, identifying variations in absorption rates at the resonator, and converting the identified variations in absorption rates into outputs related to material properties of the TUT sample.

In an embodiment, the resonator is a whispering gallery mode (WGM) silicon-based or hybrid magnetic and NIR sensitive resonator.

In an embodiment, the WGM silicon-based or hybrid resonator is coupled to a microstrip line or a dielectric waveguide.

In an embodiment, the near infrared light emitted by the emitter illuminates the WGM and microstrip line or the dielectric waveguide.

In an embodiment, the WGM silicon-based resonator is doped with aluminum.

In an embodiment, identifying variations in absorption rates at the resonator includes detecting induced changes in a magnitude and/or a phase of the transmission coefficient (S21) of the resonator.

In an embodiment, the method further includes receiving, at the receptacle, the TUT sample to be imaged further includes supporting the TUT sample on a translation stage, the translation stage configured for displacing the TUT sample.

In an embodiment, the translation stage is configured for displacing the TUT sample along a two-dimensional plane.

In an embodiment, the method further includes, prior to receiving the TUT sample, performing an assessment of the tissue imaging system by emitting, at the emitter, varying intensities of near infrared light onto the resonator.

In an embodiment, the steps of identifying variations in absorption rates at the resonator and converting the identified variations in absorption rates into outputs related to the material properties of the TUT sample are performed by a Vector Network Analyzer (VNA).

According to the present disclosure, there is further provided a non-transitory machine-readable medium (e.g., computer program products) storing machine-interpretable instruction sets which, when executed by one or more processors, cause the processor(s) to perform a method for operating a tissue imaging and biosensing system. This includes code modules operating to control the system, which can include operating characteristics, controlling emission, and potentially controlling the translation stage as described above.

The method comprises: receiving, at a receptacle, a tissue under test (TUT) sample or immunoassay membrane; positioning the TUT sample relative to a resonator and an emitter; directing, via the emitter, near-infrared (NIR) radiation toward the TUT sample in either transmission mode—wherein the NIR radiation passes through the sample and impinges on the resonator—or reflection mode—wherein the NIR radiation reflects off the sample and is re-coupled into the resonator or dielectric waveguide; detecting variations in electromagnetic absorption, scattering parameters, or resonance frequency at the sensing structure; and converting said variations into output signals indicative of one or more material, structural, or biochemical properties of the TUT sample.

In one embodiment, the system comprises a two-dimensional (2D) array of resonant structures—including whispering gallery mode (WGM) resonators or NIR-sensitive dielectric waveguides—distributed across a planar substrate. Each resonator or waveguide element is optically and electromagnetically coupled to corresponding NIR emitters and/or detectors. The array is configured to concurrently or sequentially interrogate different spatial regions of a tissue under test (TUT) sample or immunoassay membrane. By capturing local variations in signal absorption, phase shift, resonance frequency, and/or scattering parameters (e.g., S21, S11, S12), the system generates a spatially resolved diagnostic output representative of the biochemical or structural heterogeneity of the sample. This configuration enables high-resolution, real-time imaging without the need for mechanical translation stages, supporting faster acquisition times and scalable architectures for both portable and high-throughput applications.

In a further embodiment, the 2D array of resonant structure can be combined with mechanical translation to provide both options for translation, where one translation approach can be used for rough precision, and the other for fine precision. For example, mechanical translation can be used for rough translation, while electronic translation can be used for fine translation, or vice versa. Similarly, but in another variant configuration, one type of translation can be used in one axis, while the other type of translation can be used in the other axis, such that the strengths of each translation in each axis can be maximized.

The present disclosure further contemplates corresponding methods, processing workflows, and control software implementations, including those embodied in machine-readable instruction sets stored on non-transitory computer-readable media. Such control software may be configured to operate one or more components of the disclosed imaging and biosensing system, including NIR emitters, resonators or dielectric waveguides, signal detection circuitry, vector network analyzers, or processor-based modules. The control software may also perform signal acquisition, processing, spatial reconstruction, frequency-domain analysis, and generation of diagnostic or imaging outputs. In some embodiments, the software is integrated into compact or field-deployable platforms and may provide a user interface, real-time visualization, or automated result classification.

According to the present disclosure, there is further provided a tissue imaging and biosensing system comprising: a receptacle configured to receive a biological or biochemical sample, the sample comprising antigen-antibody complexes or immunoassay reagents; a resonator structure, such as a whispering gallery mode (WGM) resonator or a dielectric waveguide; and an emitter configured to direct near-infrared (NIR) radiation through the sample and onto the resonator. The system is configured to detect variations in the optical absorption, scattering parameters, or resonance characteristics at the resonator caused by specific binding events between antigens and antibodies within the sample. The system converts these variations into one or more electrical outputs indicative of the presence, concentration, or binding affinity of the antigen-antibody interactions, thereby enabling label-free, real-time immunoassay detection.

In an embodiment, the emitter is a light-emitting diode (LED) configured to emit near-infrared light.

In an embodiment, the resonator is a Whispering Gallery Mode (WGM) silicon resonator.

In an embodiment, the variations in absorption rates at the resonator are variations in a magnitude and/or phase of a transmission coefficient (S21) of the resonator.

In an embodiment, the variations in absorption rates at the resonator are variations in a magnitude and/or phase of a reverse transmission coefficient (S12) of the resonator.

In an embodiment, the variations in absorption rates at the resonator are changes in a resonant frequency of the resonator.

The tissue imaging and biosensing system described herein may be practically embodied as a compact analyzer device, which in some embodiments comprises a miniaturized integrated unit incorporating a microwave or millimeter-wave source, a corresponding detector, and one or more processors or application-specific integrated circuits (ASICs). The device may be configured to control illumination, acquire resonance data, analyze electromagnetic response characteristics, and generate diagnostic outputs. These outputs may include graphical user interface (GUI) indicators, physical or electronic alerts, data logs, or control signals for downstream systems. In particular embodiments, the system may be employed for immunoassay-based viral load detection, wherein changes in resonance parameters associated with antigen-antibody binding events are used to estimate the presence, absence, or degree of viral presence. The platform is suited for deployment in clinical, laboratory, or point-of-care environments, offering rapid, label-free, and highly sensitive detection capabilities.

The analyzer device proposed herein can also be practically integrated into more complex sensing devices that may be present, for example, in a radiology suite. The analyzer device controls the operation of the device as described herein, and generates data set outputs that can be used to generate a physical print out, for example, in respect of the tissue at test. In another variant practical embodiment, the output is a visualization being rendered on a coupled display screen or a visualization digital image that can be saved and transmitted to a user or practitioner's device. The visualization can include graphical point elements that are generated based on the X-Y translation scan of the TUT. In another variant practical embodiment, the data structure is encapsulated in the form of a data object packet that is transmitted to be incorporated into an electronic medical or an electronic health record for the individual. The approach can include X-Y imaging with raw images, with superimposed graphical features generated based on metadata of the X-Y translation scan of the TUT.

### BRIEF DESCRIPTION OF THE FIGURES

In the figures, embodiments are illustrated by way of example. It is to be expressly understood that the description and figures are only for the purpose of illustration and as an aid to understanding.

Embodiments will now be described, by way of example only, with reference to the attached figures, wherein in the figures:

FIG. 1 is a schematic illustration of an exemplary tissue imaging and biosensing system, according to some embodiments of the present disclosure. The system comprises a near-infrared (NIR) emitter configured to illuminate a tissue-under-test (TUT) sample positioned adjacent to a resonant sensing structure, such as a whispering gallery mode (WGM) resonator or a dielectric waveguide. The system further includes a receiver circuit, such as a vector network analyzer or integrated detection circuitry, configured to detect changes in the electromagnetic response of the resonator based on variations in NIR absorption or reflection, enabling real-time, label-free detection of tissue characteristics or biochemical interactions such as antigen-antibody binding

FIG. 2 is a schematic illustration and graphical depiction of a dielectric resonator for the tissue imaging system of FIG. 1, according to some embodiments of the present disclosure. The resonator may be composed of high-resistivity materials capable of supporting high-Q resonant modes, such as whispering gallery modes (WGM). Example materials include undoped or lightly doped silicon, although other near-infrared (NIR)-sensitive materials—such as gallium arsenide (GaAs), indium phosphide (InP), or germanium—may also be employed depending on the desired operating wavelength and application. The resonator is configured to interact with NIR illumination, either through transmission or reflection mode, and is operatively coupled to a signal transmission structure—such as a microstrip line or dielectric waveguide—to enable measurement of resonance characteristics (e.g., S21, S11). These measurements facilitate detection of changes in tissue composition, optical absorption, or biomolecular interactions with high sensitivity and spatial resolution.

FIG. 3 is a graphical depiction of an electromagnetic field intensity distribution associated with the dielectric resonator illustrated in FIG. 2, according to some embodiments of the present disclosure. The resonator supports high-order resonant modes, such as whispering gallery modes (WGM), characterized by strong field confinement along the periphery of the structure.

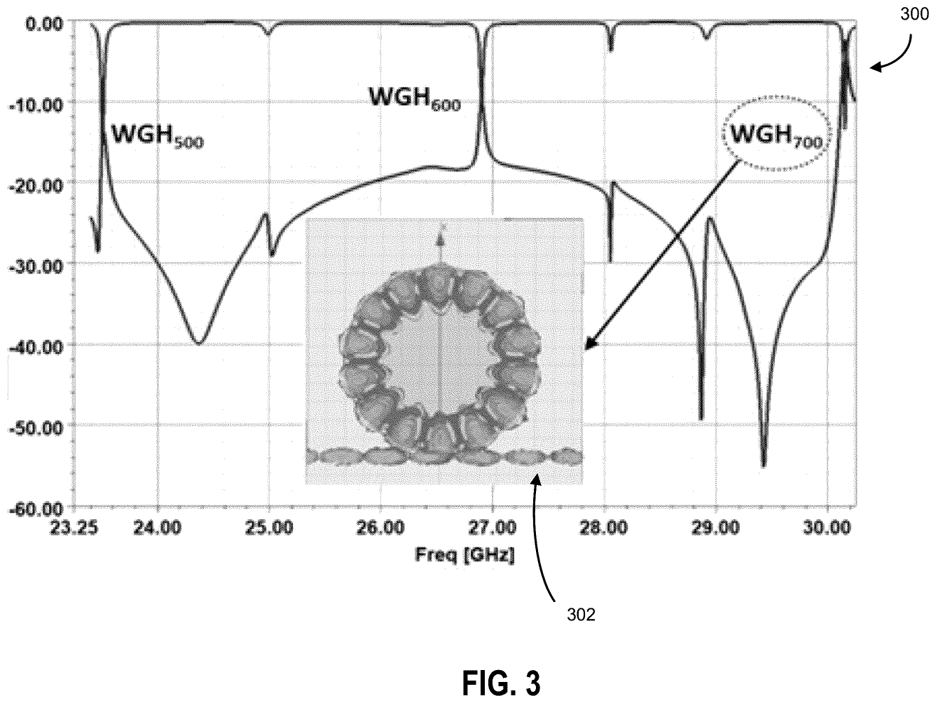

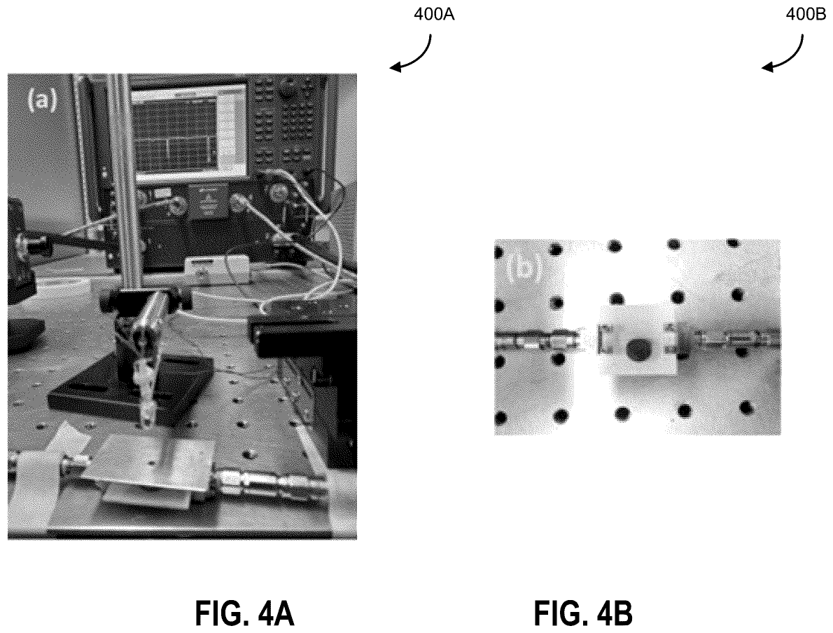

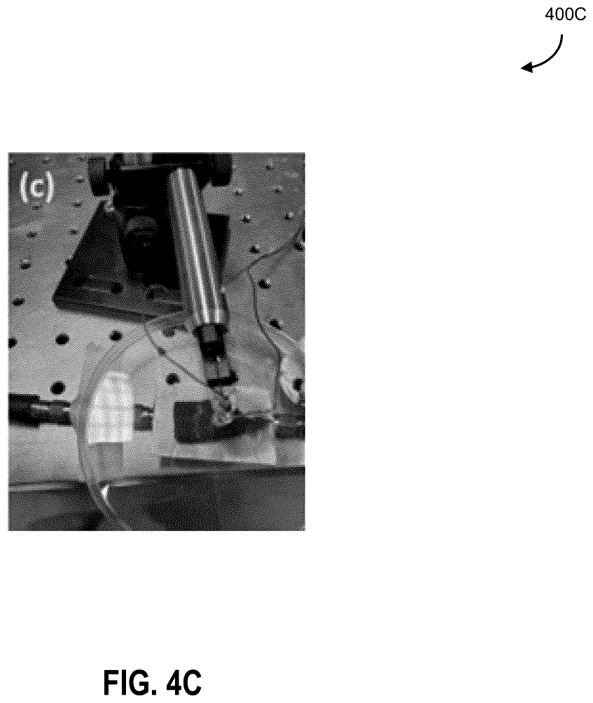

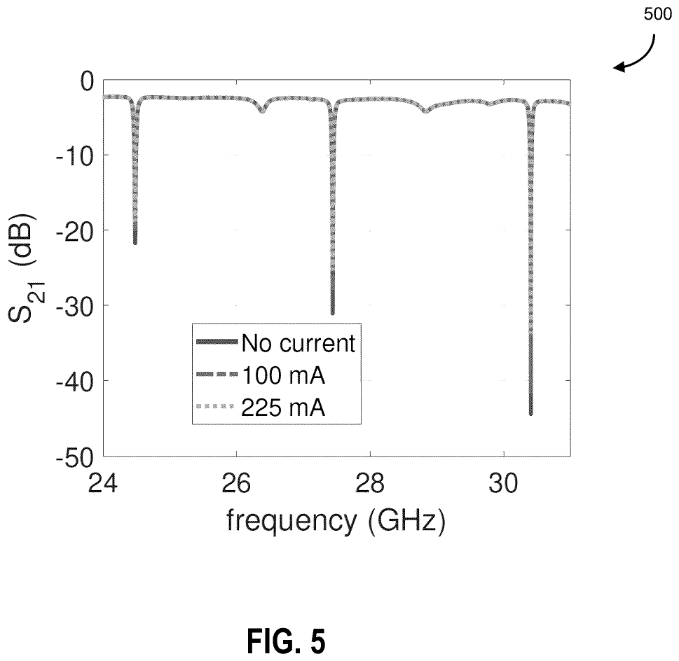

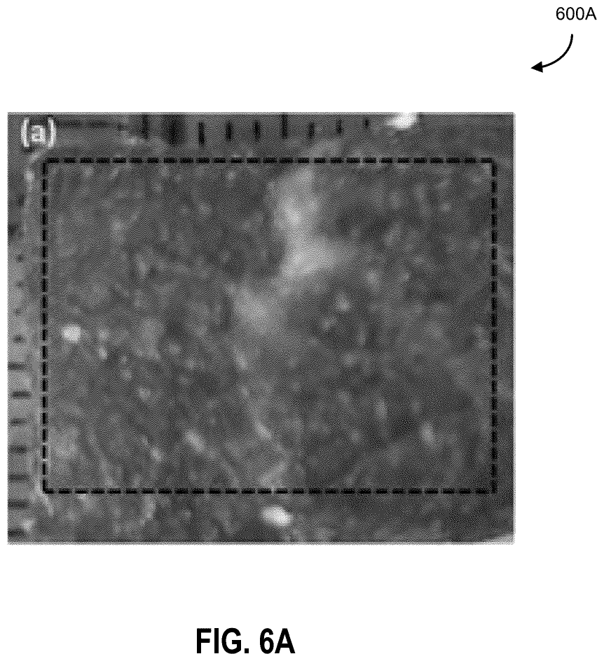

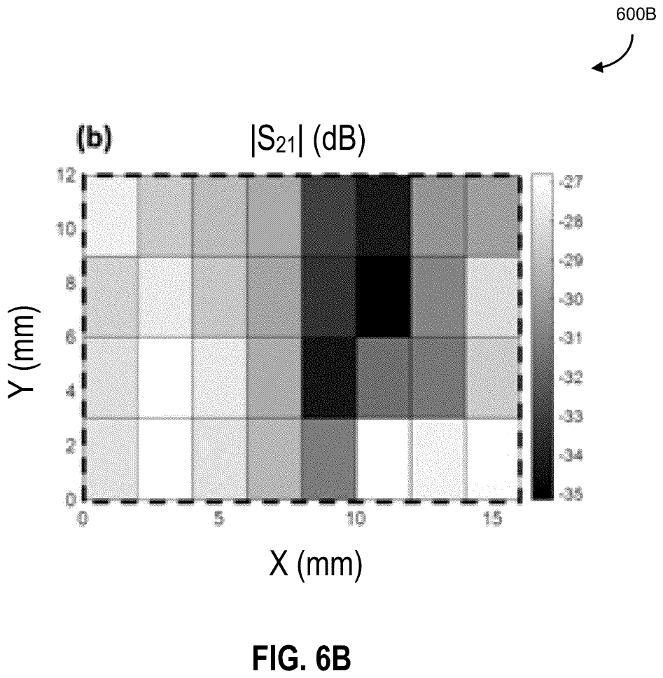

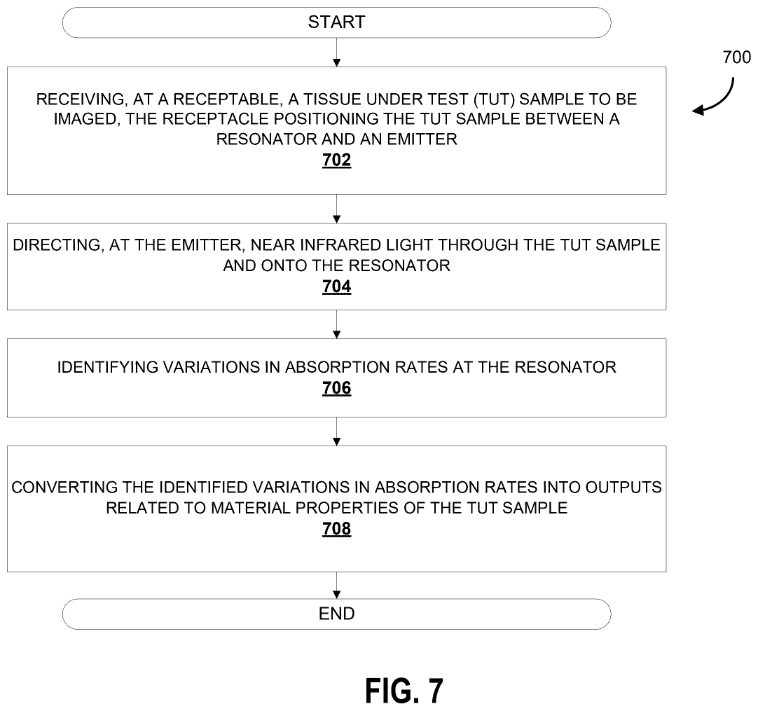

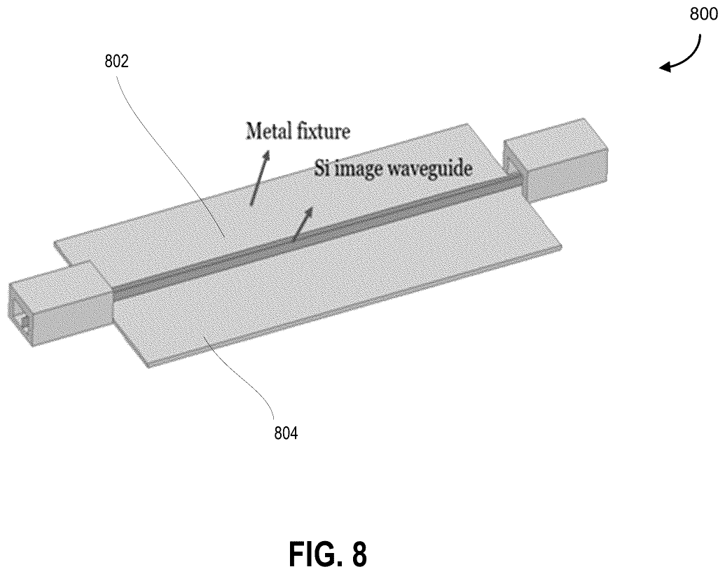

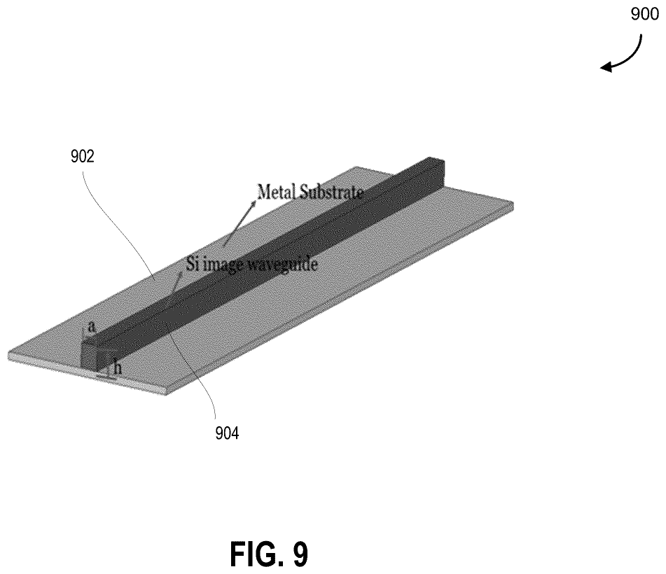

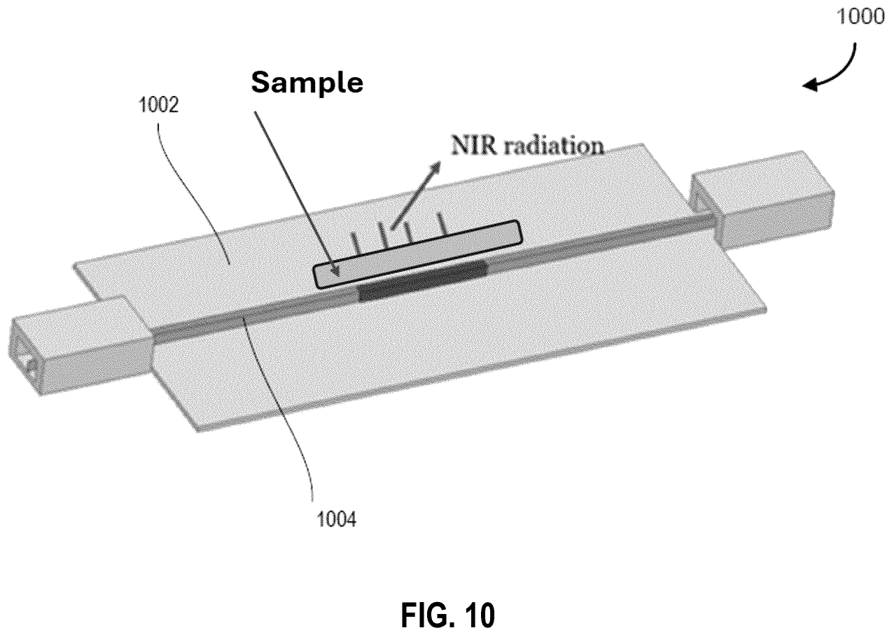

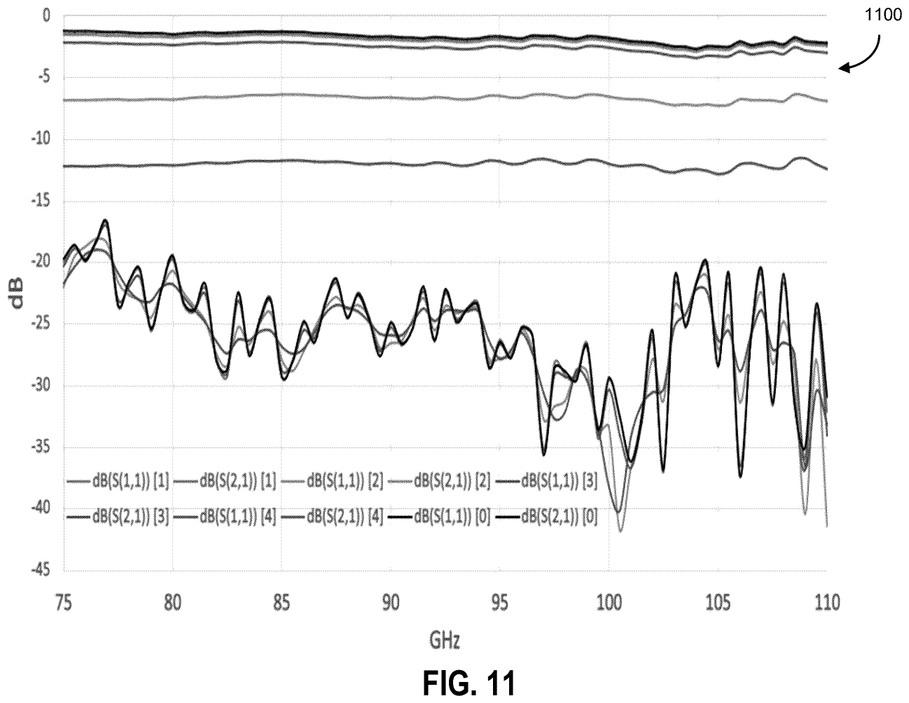

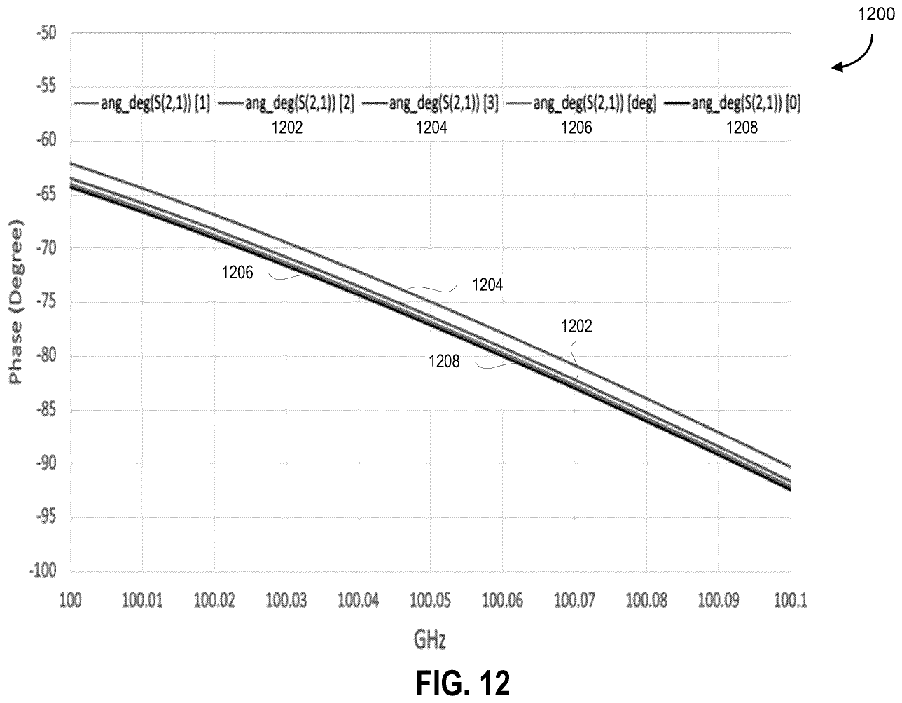

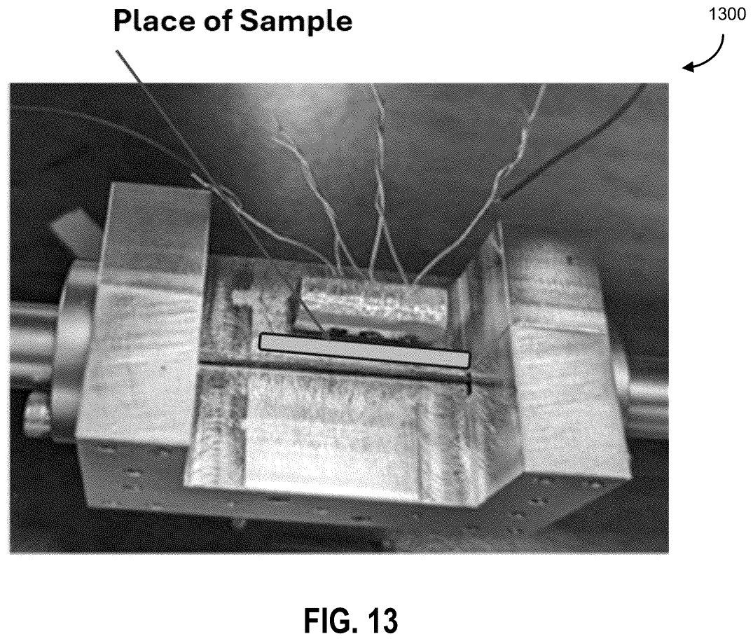

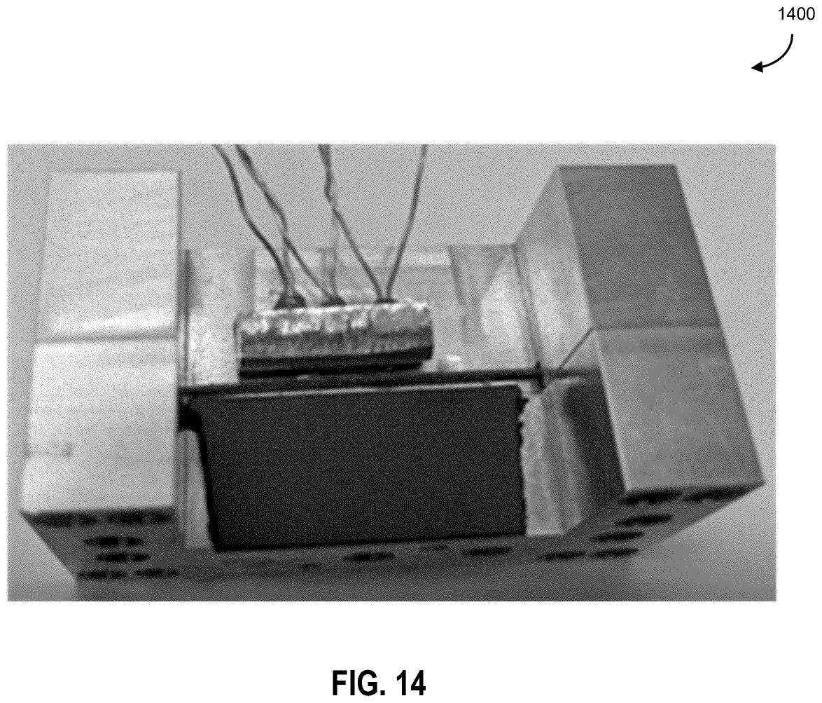

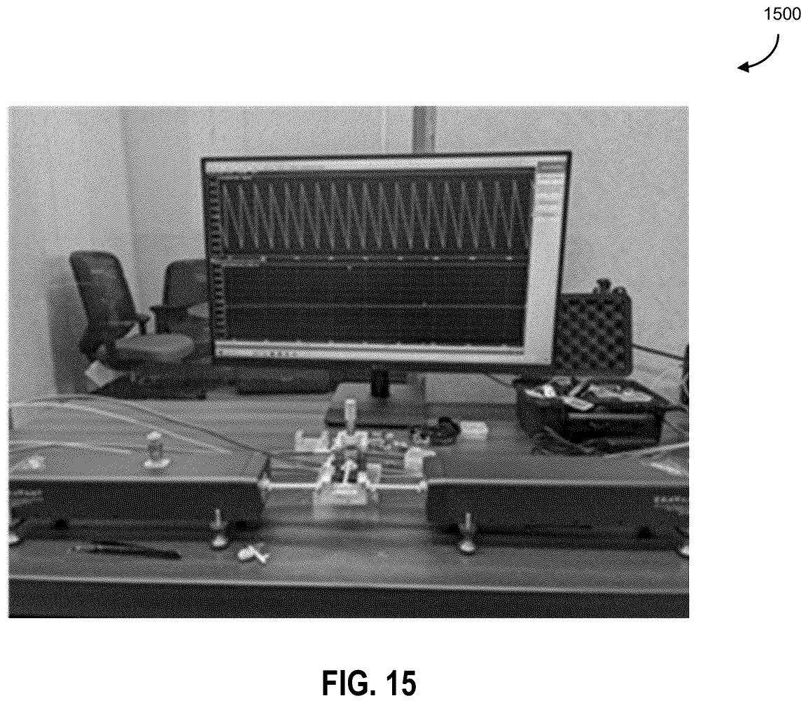

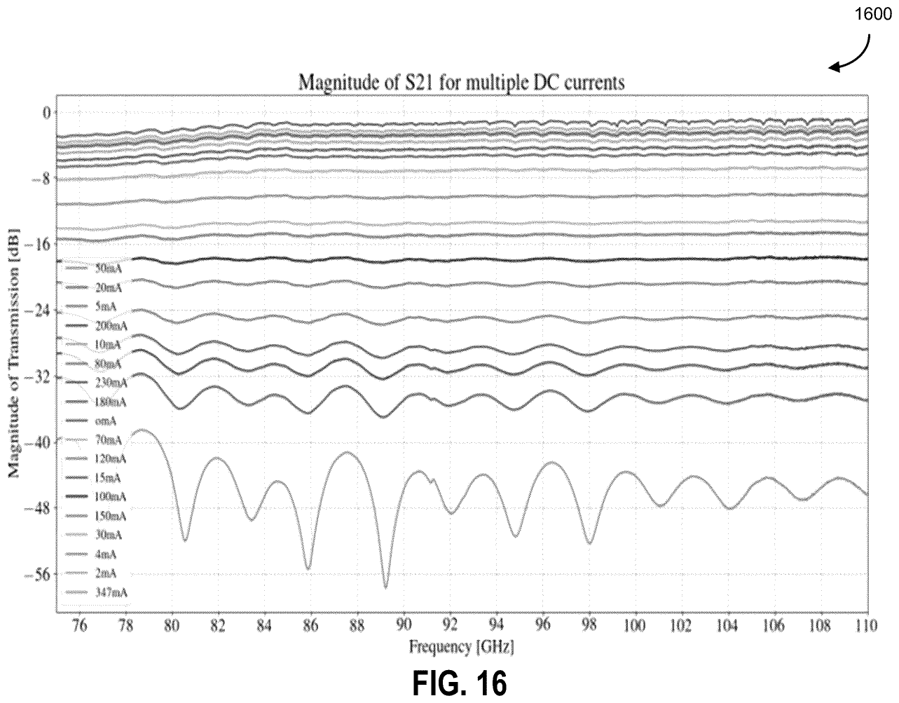

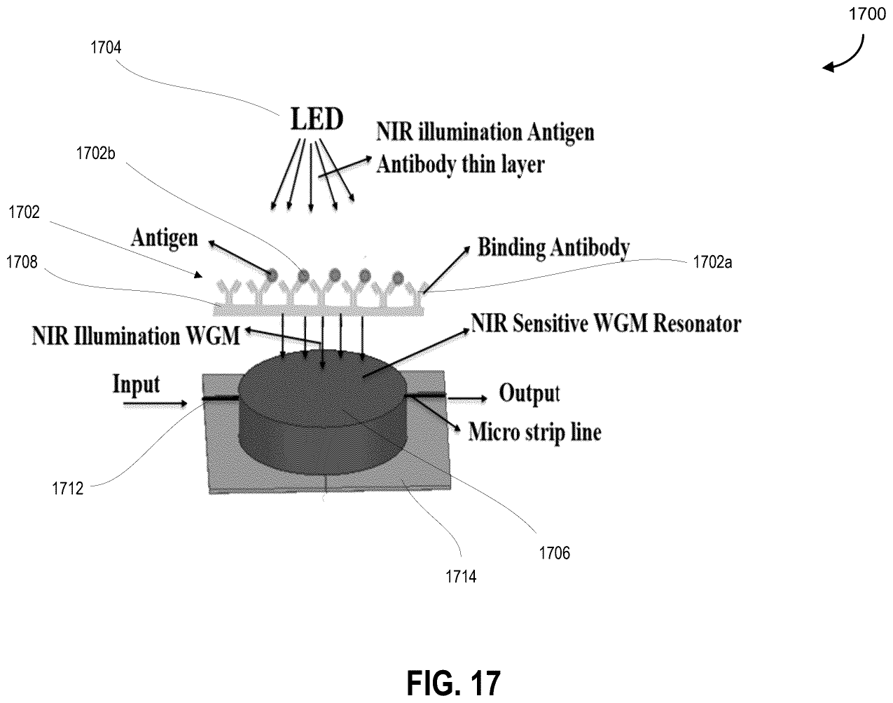

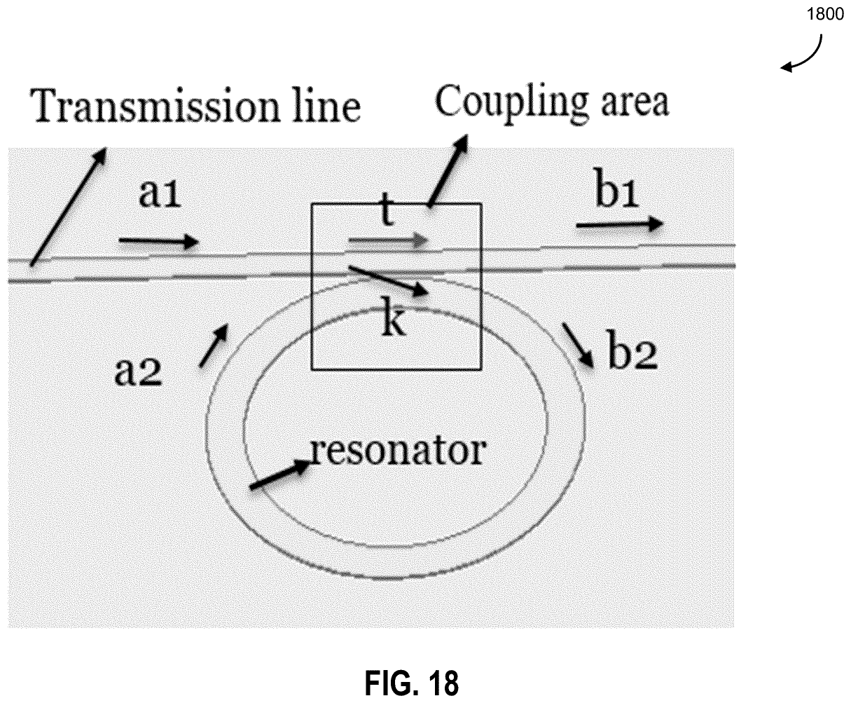

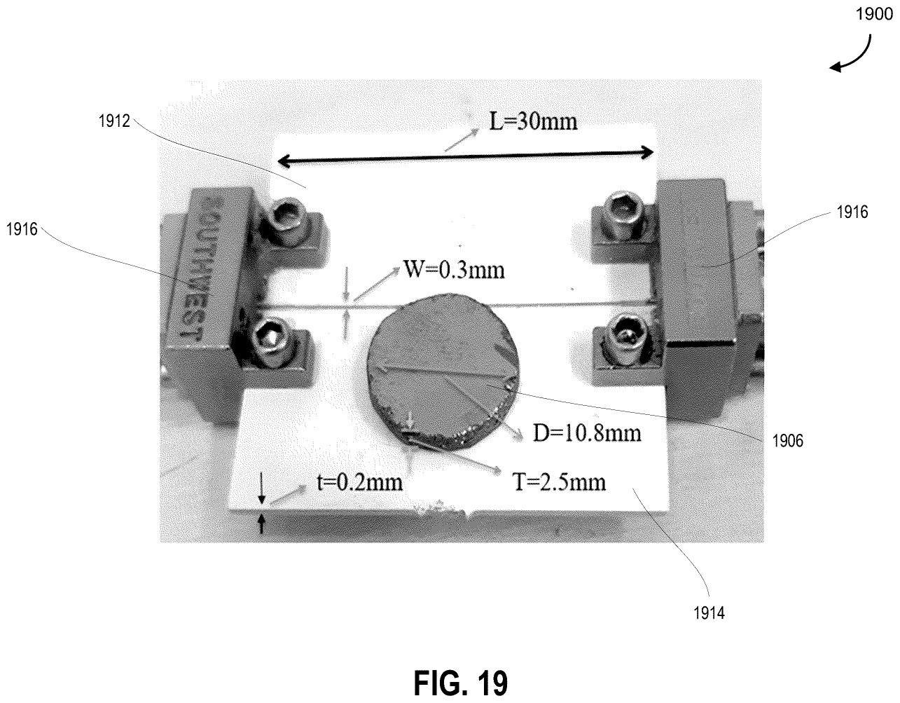

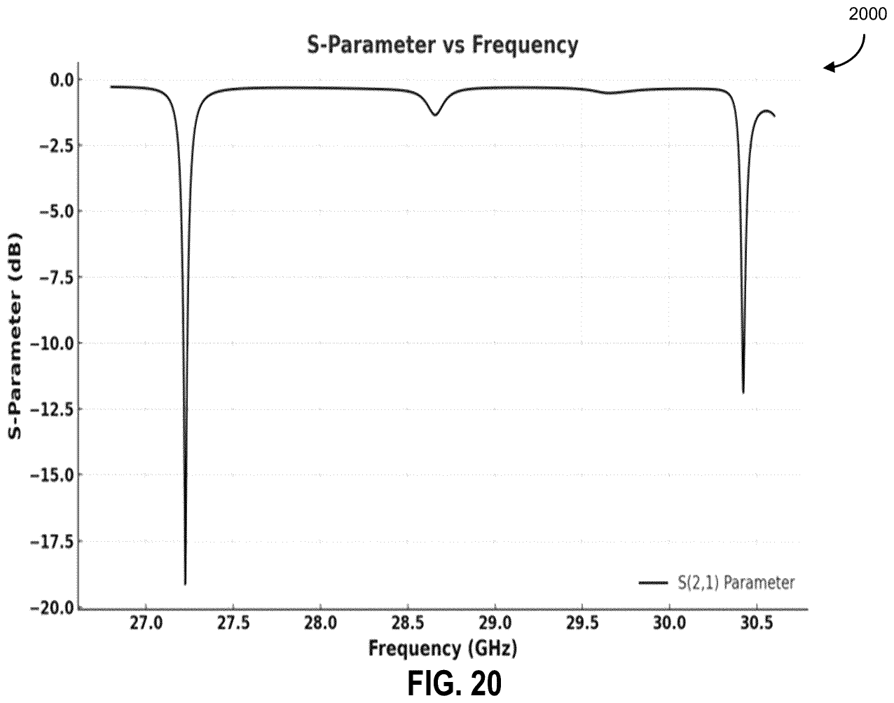

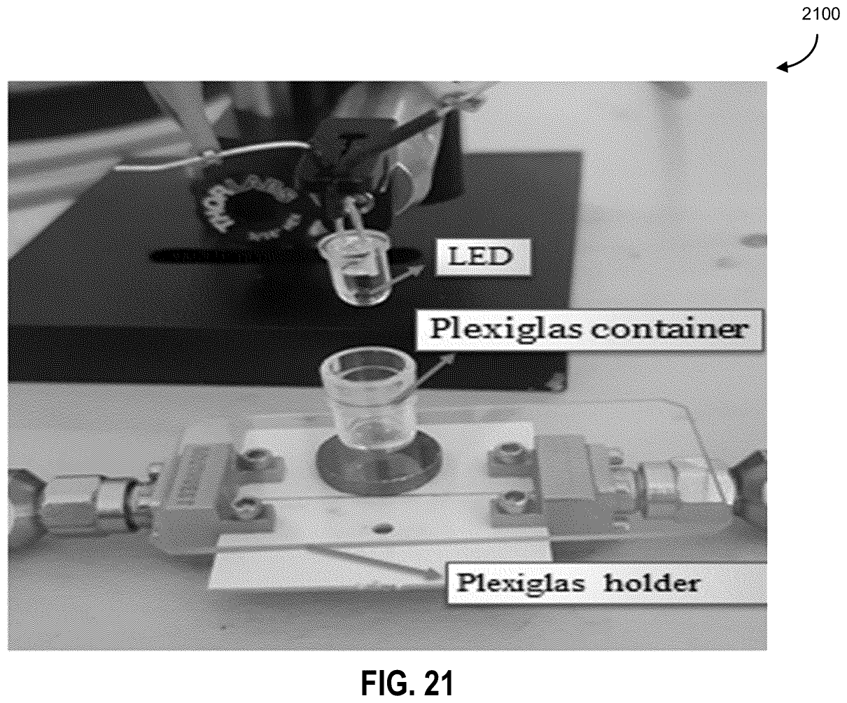

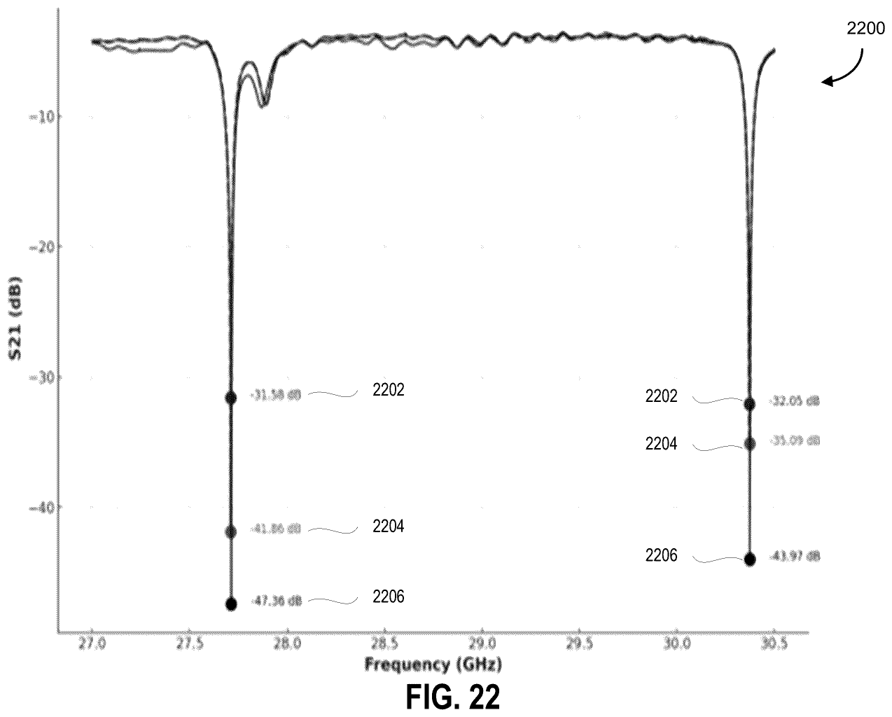

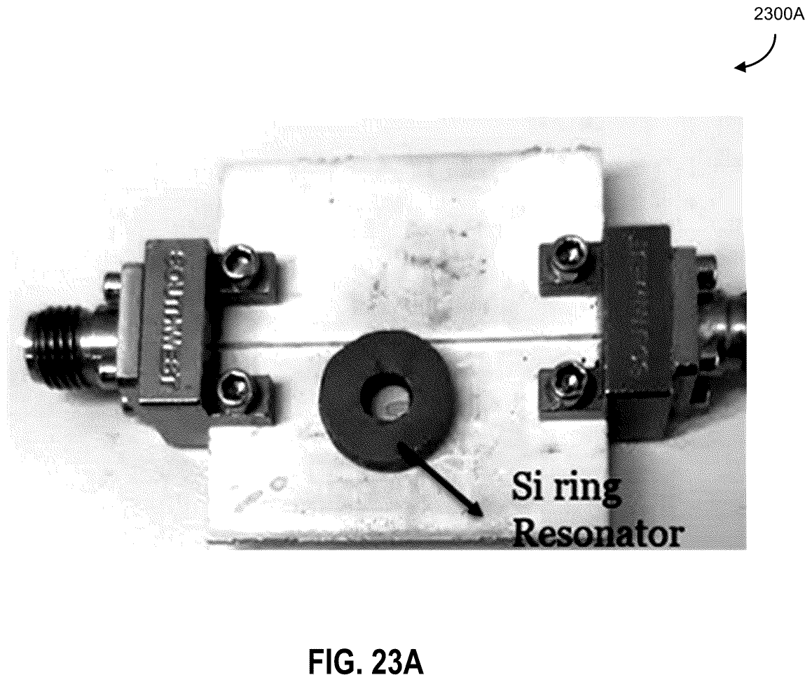

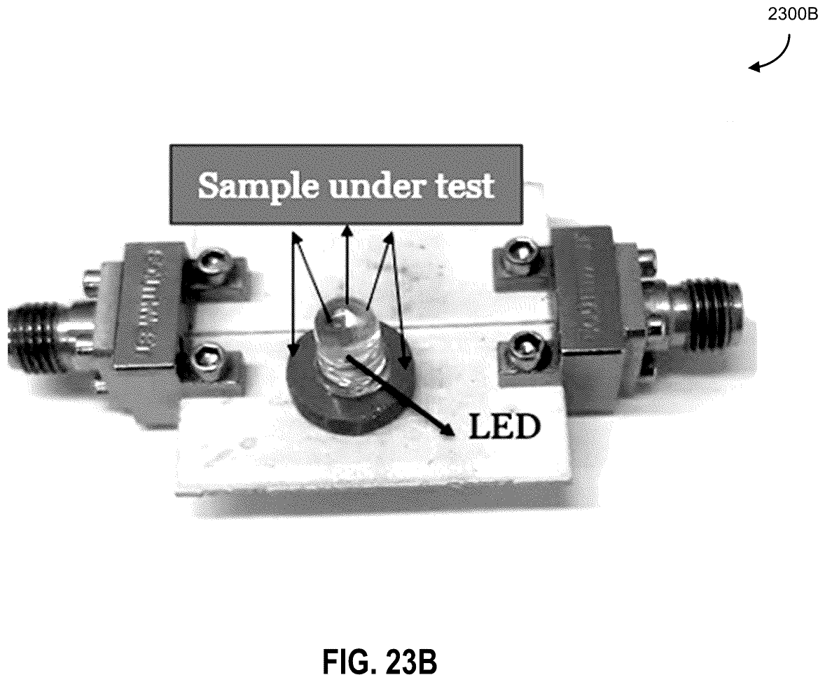

The field intensity profile shown in FIG. 3 demonstrates localized energy concentration, which enhances sensitivity to perturbations in the surrounding environment, such as changes in the optical properties of adjacent tissue samples or biomolecular films. The resonator may be fabricated from near-infrared (NIR)-sensitive materials, such as high-resistivity silicon, GaAs, InP, or germanium, to enable photoconductive modulation in response to NIR illumination. This electromagnetic response enables precise detection of absorption-induced conductivity changes and supports both transmission and reflection mode biosensing configurations.

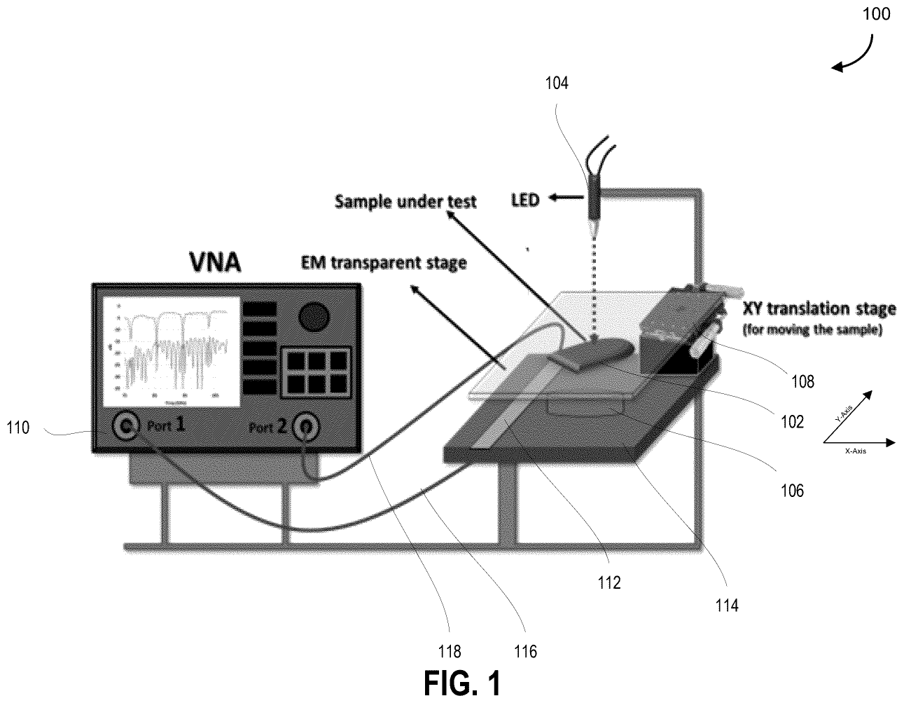

FIG. 4A is an image of the tissue imaging and biosensing system of FIG. 1, according to some embodiments. The system includes a resonator illuminated by near-infrared (NIR) light, a sample holder, and signal detection electronics. It supports both transmission and reflection modes. The resonator may be formed from NIR-sensitive materials such as high-resistivity silicon or other photoconductive dielectrics.

FIG. 4B is an image of an exemplary dielectric resonator for the system of FIG. 1, according to some embodiments. In this case, the resonator is made from high-resistivity silicon and designed to support high-order whispering gallery modes for enhanced imaging and biosensing performance.

FIG. 4C is an image of an imaging structure for the system of FIG. 1, according to some embodiments. The structure includes components for supporting near-infrared illumination and resonator or waveguide coupling, enabling transmission or reflection mode imaging.

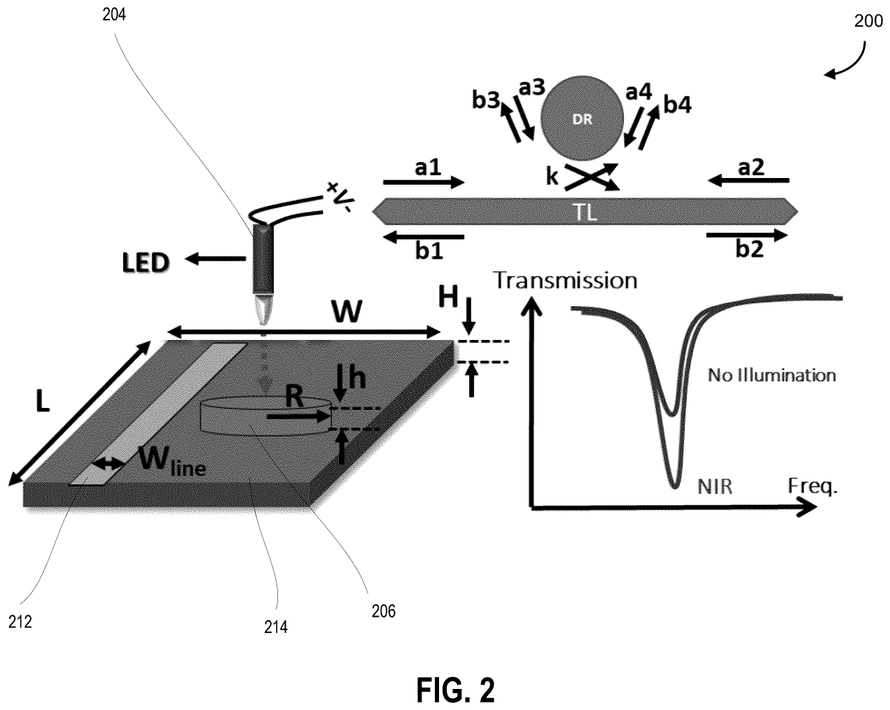

FIG. 5 is a graphical depiction of an initial experimental response of the dielectric resonator of FIG. 2 to changes in the intensity of near-infrared (NIR) light.

FIGS. 6A and 6B illustrate, respectively, a photographic image and a grayscale mapped output of a tissue under test (TUT) specimen evaluated using the system of FIG. 1. FIG. 6A presents the visible structure of the tissue, while FIG. 6B visualizes spatial variations in near-infrared (NIR) absorption, corresponding to changes in resonator response, enabling differentiation between tissue regions with distinct optical properties.

FIG. 7 is a flowchart illustrating an exemplary method for operating the tissue imaging system of FIG. 1. The method includes positioning a tissue under test (TUT) sample, illuminating it with near-infrared (NIR) light, detecting variations in resonator response, and generating output data corresponding to material or biochemical properties of the TUT sample.

FIG. 8 is a perspective view of a variable attenuator incorporating a dielectric waveguide fabricated from a near-infrared (NIR) sensitive material-such as high-resistivity silicon or chalcogenide glass-configured to modulate electromagnetic signal transmission in response to incident NIR illumination. The waveguide structure may include rib or ridge geometries optimized for efficient coupling and tunable attenuation, according to some embodiments.

FIG. 9 is a perspective cross-sectional view of a silicon-based dielectric waveguide image guide, configured for near-infrared (NIR) sensitivity, as used in the variable attenuator of FIG. 8. The structure demonstrates internal light propagation and modulation paths through high-resistivity silicon or similar NIR-absorptive materials, enabling attenuation control via optical input.

FIG. 10 is a perspective view of a backside illumination configuration for the variable attenuator of FIG. 8. A sample can be positioned between the near-infrared (NIR) illumination source and the NIR-sensitive dielectric waveguide, allowing modulation of the waveguide's properties based on sample-induced absorption.

FIG. 11 is a graphical depiction of exemplary simulation results demonstrating the variable attenuation mechanism of the sensor of FIG. 8 under varying near-infrared (NIR) irradiation intensities.

FIG. 12 is a graphical depiction of exemplary of simulation results of FIG. 11 for the phase of S21 or transmission.

FIG. 13 is an image of an exemplary variable attenuator in a fixture, according to some embodiments and potential placement of sample between the LED array and the dielectric waveguide.

FIG. 14 is an image of the variable attenuator in a fixture of FIG. 13 with an exemplary microwave absorber sheet.

FIG. 15 is an image of an exemplary measurement set up for the NIR illuminated variable attenuator of FIG. 8.

FIG. 16 is a graphical depiction of exemplary measurement results from the measurement set up of FIG. 15 versus intensity of the NIR light.

FIG. 17 is a schematic illustration of another exemplary biosensing system, according to some embodiments.

FIG. 18 is a schematic illustration of the dielectric resonator for the biosensing system of FIG. 17, according to some embodiments.

FIG. 19 is an image of an exemplary dielectric resonator for the system of FIG. 17, according to some embodiments.

FIG. 20 is a graphical depiction of exemplary transmission coefficient parameters for the dielectric resonator of FIGS. 18-19 based on simulation.

FIG. 21 is an image of an exemplary setup for the biosensing system of FIG. 17.

FIG. 22 is a graphical depiction of an experimental magnitude of transmission for the dielectric resonator of FIGS. 18-19 in response to changes in Near Infrared Light for different bio-samples.

FIG. 23A is an image showing the Si ring resonator, according to some embodiments.

FIG. 23B is an image showing a sample under test, as well as a coupled indicator LED, according to some embodiments.

### DETAILED DESCRIPTION

The present disclosure provides systems and methods for tissue imaging and biochemical sensing. While the embodiments described herein primarily reference biological applications—such as the analysis of human or animal tissues—this is intended solely for illustrative purposes and not as a limitation. The disclosed systems are equally applicable to non-biological specimens, including but not limited to polymers, composites, and industrial materials, enabling a broad range of imaging and material characterization use cases.

Various efforts have been directed toward addressing the limitations associated with conventional imaging modalities, such as X-ray mammography, which are constrained by factors including ionizing radiation exposure, limited contrast resolution, and procedural invasiveness. Among proposed alternatives, microwave imaging technologies have emerged as promising candidates for non-invasive diagnostic applications, including but not limited to breast and brain tissue imaging. These technologies leverage non-ionizing electromagnetic fields to enable deeper tissue penetration and improved safety profiles.

Microwave imaging technologies provide several advantageous characteristics, including the use of non-ionizing radiation, system compactness, and potential for cost-effective deployment. Nonetheless, certain technical limitations remain, particularly in relation to spatial resolution, signal-to-noise performance, and clutter rejection. These limitations have been documented in the literature, such as in D. Tajik, J. Trac, and N. K. Nikolova, “Quality Control of Microwave Equipment for Tissue Imaging,” IEEE Journal of Electromagnetics, RF and Microwaves in Medicine and Biology, vol. 4, no. 1, pp. 52-60, March 2020, and in X. Lin, Y. Ding, Z. Gong, and Y. Chen, “Hybrid Microwave Medical Imaging Approach Combining Quantitative and Qualitative Algorithms,” IEEE Antennas and Wireless Propagation Letters, vol. 20, no. 4, pp. 438-442, April 2021. Accordingly, further enhancements are necessary to improve image clarity, diagnostic confidence, and robustness under practical deployment conditions.

To address the limitations inherent in conventional microwave imaging systems, recent research has explored the use of Near Infrared (NIR) spectral bands—specifically within the range of approximately 650 nanometers (nm) to 950 nm—as a complementary or alternative modality for tissue diagnostics. NIR imaging provides deeper tissue penetration and reduced scattering compared to visible light, while maintaining non-ionizing characteristics. Notable investigations into the diagnostic utility of NIR include L. A. Sordillo et al., “Deep optical imaging of tissue using the second and third near-infrared spectral windows,” Journal of Biomedical Optics, vol. 19, no. 5, 056004, May 2014; P. K. Upputuri and M. Pramanik, “Photoacoustic imaging in the second near-infrared window: a review,” Journal of Biomedical Optics, vol. 24, no. 4, 040901, April 2019; and B. Zhu et al., “Comparison of NIR Versus SWIR Fluorescence Image Device Performance Using Working Standards Calibrated With SI Units,” IEEE Transactions on Medical Imaging, vol. 39, no. 4, pp. 944-951, April 2020. These works demonstrate the potential for improved imaging contrast, signal-to-noise performance, and tissue differentiation within NIR spectral windows.

The Near Infrared (NIR) spectral range has demonstrated significant promise for advancing imaging performance and broadening diagnostic capabilities. As NIR light propagates through biological tissue, its behavior is governed by complex interactions involving absorption, scattering, and reflection phenomena. These interactions are influenced by the wavelength-dependent optical properties of the tissue, enabling differential contrast and depth profiling that are particularly suited for non-invasive biomedical imaging applications.

The use of Near Infrared (NIR) spectroscopy has demonstrated utility in the optical characterization of various physiological and structural components within human tissue. This includes applications such as the quantification of oxygen saturation levels in cerebral and muscular tissues, as well as the identification of biochemical constituents based on their absorption spectra. Prior studies, including those by Ferrari and Quaresima (2012) and by Philip, Paul, and Chappell (2012), have highlighted the effectiveness of NIR spectroscopy in capturing spatial and compositional information through non-invasive, label-free modalities.

In exemplary implementations, a typical Near Infrared (NIR) spectrometer comprises a continuous-wave NIR light source configured to irradiate a target tissue sample, and a corresponding NIR detector configured to capture the interaction of photons with the tissue. As the NIR photons propagate through the tissue, they undergo various optical phenomena including transmission, scattering, and reflection. These modified photons are subsequently collected, for example via optical fibers, and transmitted to a spectrometer for spectral analysis. The sensitivity of the NIR receiver is critical in achieving high detection resolution, particularly when analyzing heterogeneous or multilayered tissues. By way of illustration, Raeesi et al. (2020) disclose an ultra-sensitive NIR spectrometer employing a silicon-based Whispering Gallery Mode (WGM) resonator, which demonstrated enhanced performance for glucose detection through precise resonance modulation in response to NIR illumination.

As disclosed in M. Neshat et al., “Traveling-wave whispering gallery resonance sensor in millimeter-wave range,” Electronics Letters, vol. 44, no. 17, pp. 1020-122 August 2008, and in M. Neshat et al., “Whispering gallery mode resonance sensor for dielectric sensing of drug tablets,” Measurement Science and Technology, vol. 21, no. 1, pp. 015202 (1)-015202 (11), 2010, the Whispering Gallery Mode (WGM) technique offers significant advantages in terms of sensing accuracy, sensitivity, and resolution. These characteristics make WGM-based resonators highly suitable for a wide range of applications, including dielectric characterization, pharmaceutical quality control, and material analysis. The intrinsic properties of WGM structures—such as high-quality factors (Q-factors) and confinement of electromagnetic energy within the resonator periphery—contribute to their exceptional precision in detecting minute perturbations caused by changes in the surrounding medium or analyte interaction.

Referring now to FIG. 1, there is shown a schematic illustration of an exemplary tissue imaging and sensing system 100, in accordance with an embodiment of the present disclosure. The system 100 comprises a tissue under test (TUT) sample 102, which is positioned between a near-infrared (NIR) light-emitting diode (LED) emitter 104 and a dielectric resonator (DR) 106. The TUT sample 102 is supported on a receptacle 108 configured to receive and hold the sample during imaging and sensing operations. In some embodiments, the receptacle 108 comprises a transparent translation stage, which is configured to displace the TUT sample 102 along a two-dimensional plane, thereby facilitating spatially-resolved scanning. The DR 106 is configured to detect variations in absorption of the NIR radiation resulting from interactions with the TUT sample 102. These variations induce measurable changes in the electromagnetic response of the DR 106, which are converted into outputs indicative of the material properties of the sample. The system further includes a vector network analyzer (VNA) 110 operatively coupled to the DR 106 to monitor relevant parameters (e.g., S-parameters), although alternative analysis modules or circuits may be employed in other embodiments.

In the illustrated embodiment, the dielectric resonator (DR) 106 comprises a whispering gallery mode (WGM) silicon-based resonator, although other embodiments may employ different infrared-sensitive resonator materials or a hybrid of magnetic and NIR sensitive material for non-reciprocal sensing mechanism. Accordingly, the DR 106 may also be referred to as a WGM resonator 106. In one embodiment, the WGM resonator 106 is doped with aluminum to enhance its sensitivity to near-infrared (NIR) illumination, functioning as an ultra-sensitive NIR receiver. The WGM resonator 106 is operatively coupled to a microstrip transmission line 112, and both components are supported on a platform 114 or equivalent substrate.

During operation, NIR radiation emitted by the emitter 104 interacts with the tissue under test (TUT) sample 102 and subsequently illuminates the WGM resonator 106 and microstrip line 112. The resonator's electromagnetic response varies depending on the transmission, reflection, and scattering characteristics of the NIR signal influenced by the TUT sample 102. These interactions cause measurable changes in transmission parameters, particularly in the magnitude and phase of the S21 scattering parameter, which characterizes signal transmission between ports in the system. Such variations, detected in the microwave or millimeter-wave frequency domain, provide insight into the optical and dielectric properties of the TUT sample 102.

The microstrip line 112 is electrically connected to a vector network analyzer (VNA) 110 via end-launch connectors 116 and 118 affixed to opposite ends of the microstrip. Alternative connection mechanisms or analysis modules may be employed in other embodiments.

Referring now to FIG. 2, a schematic illustration and graphical depiction 200 of the dielectric resonator (DR) 206 for the tissue imaging system of FIG. 1 is shown, in accordance with some embodiments. As previously described, the DR 206 is positioned on a supporting platform 214 and is operatively coupled to a microstrip transmission line 212 to enable an ultra-sensitive detection configuration. This configuration is exposed to near-infrared (NIR) illumination from the emitter 204 through a tissue under test (TUT) sample (not shown in FIG. 2 for clarity). In certain embodiments, the DR 206 may alternatively be coupled to a dielectric waveguide formed from NIR-sensitive materials to enhance optical-electromagnetic interaction.

The illustrated arrangement corresponds to a four-port circuit model, which facilitates the analysis of energy coupling between the resonator and waveguide or transmission line. This model is based on foundational work presented in A. Yariv, “Universal relations for coupling of optical power between micro-resonators and dielectric waveguides,” Electronics Letters, vol. 36, no. 4, pp. 321-322, 2000, which defines the scattering parameters (S-parameters) for such resonant systems. These parameters govern the behavior of input-output signal relationships across the coupled ports and form the basis for extracting diagnostic information from the resonator's electromagnetic response.

$${b}_{2} = \frac{\sqrt{1 - {k}^{2}} - exp ( - α )}{1 - \sqrt{1 - {k}^{2}} exp ( - α )} {a}_{1} \qquad ( 1 )$$

$${b}_{4} = \frac{jk}{1 - \sqrt{1 - {k}^{2}} exp ( - α )} {a}_{1} \qquad ( 2 )$$

$$T = \frac{1 + {α}^{2} - {k}^{2} - 2 α \sqrt{1 - {k}^{2}} cos ( θ )}{1 + {α}^{2} - {( α k )}^{2} - 2 α \sqrt{1 - {k}^{2}} cos ( θ )} \qquad ( 3 )$$

where, a₁, k, and T denote the input power, the coupling coefficient, and the transmission coefficient respectively. A power exchange transpires within the coupling region. The loss factor, α (0<α<1), also known as the resonator's 206 circulation factor, impacts the results of this power exchange. For instance, when the resonator 206 has a circulation factor α of 1, this implies near losslessness with an ultrahigh Q factor. In the above equations, θ represents phase shift after traversing a rotation distance of 2Tr around the resonator 206, where r represents the radius of the DR 206. In operation, a resonant state materializes when θ=2 mm and α=1-k, leading to nearly zero transmission power (T≈0), also referred to as a near-critical coupling condition.

In various embodiments, a transmission line or dielectric waveguide is operatively coupled with a resonator. The quality factor (Q-factor) of the resonator is highly sensitive to the intensity of incident near-infrared (NIR) radiation. Variations in NIR illumination incident upon the resonator result in corresponding perturbations in the resonator's electromagnetic response at microwave, millimeter-wave, or sub-terahertz frequencies. These perturbations—manifesting as shifts in transmission characteristics such as amplitude, phase, or resonance frequency—are detected with high precision, wherein the sensitivity is proportional to the resonator's Q-factor. Accordingly, the proposed system functions as a high-performance, sensitive NIR detector integrated into an imaging platform, offering significant improvements over conventional detection and imaging approaches. The resonator material, geometry, and structural configuration may be selected or tuned based on application-specific requirements, including but not limited to biological imaging, immunoassay sensing, or non-destructive material evaluation.

The system 100 is configured to operate near the critical coupling regime, wherein the transmission characteristics of the resonator become highly sensitive to perturbations in the internal loss factor (a). Modulation of the loss factor induces substantial changes in output transmission, thereby enabling fine-grained detection of analyte-induced variations. As demonstrated in A. E. Omer, S. Gigoyan, G. Shaker, and S. Safavi-Naeini, “Whispering-gallery-mode microwave sensing platform for oil quality control applications,” IEEE Internet of Things Journal, vol. 9, no. 6, pp. 4065-475 March 2022, small perturbations in a under resonant conditions can lead to pronounced phase shifts at discrete frequency points. In the present system, as the near-infrared (NIR) radiation emitted from emitter 104 or 204 propagates through the tissue under test (TUT) sample 102 and impinges upon the dielectric resonator (DR) 106 or 206, the resulting interaction modulates the resonator's electromagnetic response, inducing measurable variations in the transmission coefficient (S21). Operating in proximity to the critical coupling condition allows even subtle changes in NIR absorption or scattering to manifest as discernible shifts in signal magnitude or phase, thereby enabling high-sensitivity detection. The DR 106, 206 is thus engineered to support well-defined whispering gallery mode (WGM) resonances within a target frequency band. In one exemplary implementation, the design of the DR 106, 206 is guided by the Effective Dielectric Constant (EDC) approach and further validated via full-wave electromagnetic simulations to ensure optimal resonant performance under operational conditions.

A variety of near-infrared (NIR)-sensitive materials may be employed for constructing the dielectric resonator (DR) 106, 206. The material selection is based, in part, on the alignment between the photon energy of the incident NIR illumination and the band gap energy of the semiconductor material used in the resonator. In one exemplary embodiment, silicon (Si) is selected as the NIR-responsive material due to its band gap energy of approximately 1.12 eV at room temperature, which is compatible with the energy of NIR photons. To enhance NIR absorption and optimize photonic interaction, the silicon used in the DR 106, 206 may be doped with group III elements to slightly modulate its band gap characteristics. In the depicted embodiment, the DR 106, 206 is composed of aluminum-doped silicon, providing enhanced NIR sensitivity. The resonator is further configured to support whispering gallery mode (WGM) resonance of the 7th order within the Ka-band frequency range. In one implementation, the WGM structure features a radius of R=5.13 mm and exhibits a resonant frequency centered around 30.41 GHZ. Full-wave electromagnetic simulations confirm the spatial field confinement and mode distribution of the DR 106, 206 under these conditions, as further detailed in reference to FIG. 3 in the graph 300.

Still referring to FIG. 2, an exemplary microwave-NIR hybrid structure 200 is depicted. In the illustrated embodiment, the structure comprises a microstrip transmission line 212 operatively coupled to a whispering gallery mode (WGM) dielectric resonator 206. Near-infrared (NIR) illumination is incident upon the resonator 206 from above, provided by a NIR-LED emitter 204. This configuration facilitates resonant excitation and sensing through the interaction of the NIR-modulated dielectric environment and the microwave transmission path. Representative design parameters for the microstrip line 212, resonator 206, and related components—as exemplified in the schematic of FIG. 2—are summarized below in Table I.

TABLE I
DESIGN PARAMETERS OF THE
DR-MICROSTRIP STRUCTURE

| Parameter | L | W | H | Wₗᵢₙₑ | t | R | h |
|---|---|---|---|---|---|---|---|
| Values (mm) | 30 | 20 | 0.2 | 0.28 | 0.017 | 5.13 | 1.44 |

where L, W and H are the length, width, and height respectively, of the exemplary rectangular-based platform 214, Wₗᵢₙₑ is the width of the microstrip line 212, R is the radius of the illustratively circular-based resonator 206, h is the height of the resonator 206, and t is the thickness of the microstrip line 212. Other dimensions and shapes for these components may be contemplated. In various embodiments, the microstrip line 212 is designed using the Rogers 4360G2 substrate with a permittivity of 6.15, a loss tangent of 0.0038, and a thickness of 200 μm. Other substrate parameters may be contemplated. Also shown in FIG. 2 is a schematic depiction of the dielectric resonator DR and a transmission line TL, with various incident and reflected powers a1-a4, k, b1-b4 to and from each port of the transmission line TL.

FIG. 3 presents a graphical depiction 300 of the electromagnetic field intensity distribution for the dielectric resonator 206 of FIG. 2. An eigenmode solution is extracted to validate analytical predictions of resonance behavior. As shown, the insertion loss and return loss characteristics of the WGM resonator 206—when coupled to the microstrip line 212—are revealed across the 23.25-30.25 GHz frequency range, with three dominant WGM resonances emerging. Variations in NIR intensity modulate the absorption loss, inducing shifts in both the effective dielectric constant and the conductivity of the resonator structure. These effects are demonstrated via full-wave electromagnetic simulations. The resulting variations in the transmission coefficient S21 under NIR illumination are particularly observable in targeted regions of the Ka-band. Embedded in the figure is image 302, illustrating the normalized Ez field component for the WGH700 mode within the XY plane of the DR 206.

As discussed with reference to FIG. 1, the tissue under test (TUT) sample 102 is positioned between the NIR LED emitter 104 and the dielectric resonator (DR) 106. In the depicted embodiment, a precise translation stage—serving as the receptacle 108—enables controlled two-dimensional displacement of the TUT sample 102. The sample thickness may be selected to ensure sufficient transmission of NIR light. Incremental translation of the TUT sample 102 along the X and Y axes, while maintaining the fixed position of the emitter 104, results in spatially varying NIR intensities reaching the DR 106. These variations, influenced by the tissue's local optical properties, induce corresponding fluctuations in the S21 transmission coefficient (as demonstrated in FIG. 3). This relationship between absorbed light intensity and S-parameter variations underpins the imaging methodology disclosed herein.

Referring now to FIGS. 4A-4C, exemplary images 400a, 400b, and 400c illustrate an embodiment of the system 100 of FIG. 1. Image 400a displays the DR 106 and microstrip line 112 illuminated by the NIR LED emitter 104, along with the connected VNA 110. Image 400b provides a close-up of the DR 106 and microstrip line 112, while image 400c offers an enhanced view of the LED emitter 104 positioned above the resonator setup. In the illustrated configuration, a high-resistivity, aluminum-doped silicon WGM DR 106 is positioned adjacent to a 50-ohm microstrip line 112. The DR 106 is illuminated from above by an 870 nm NIR LED emitter 104, fixed at a vertical distance of 10 cm. This setup serves as an illustrative example; variations in component placement, material, and geometry are also contemplated.

Referring now to FIG. 5, graphical depiction 500 illustrates an exemplary initial experimental phase for the dielectric resonator 106, 206. This phase, which may be optional, involves system assessment without a TUT sample 102. During this evaluation, varying intensities of NIR light are emitted from the LED emitter 104, and corresponding S21 measurements are recorded. FIG. 5 presents S21 responses for the WGH700 resonance mode under different NIR light intensities. The results highlight the sensitivity of resonance depth to NIR intensity, with near-critical coupling observed when the emitter operates at V=200 mV and I=225 mA. Other operating values may also be applicable.

Referring now to FIGS. 6A-6B, an image 600a of the TUT sample 102 and a corresponding black and white mapped image 600b of the TUT sample 102 at 30.4 GHz are shown. Subsequently to the above-described experimental phase, the TUT sample 102 is introduced to the receptacle 108, for instance by being placed on a transparent fixture. This fixture is maneuverable horizontally along both the X and Y axes. Maneuvering of the fixture can be facilitated by an optional precision micrometer translation stage. In an exemplary embodiment, the collective imaging scope spans a 16×12 mm² area. The visual representation 600a of the TUT sample 12 testing region, depicted in FIG. 6A, is matched with the corresponding mapped black/white image 600b generated through the measurement of S21 at 30.4 GHz in distinct locations, as illustrated in FIG. 6B. These findings distinctly differentiate between the regions of standard tissue composition and areas with higher fat content within the animal flesh specimen. The variation in readings of the depth of S21 is proportional to the variation in the TUT sample 102. In addition to the depth of resonance in S21, the phase of S21 may also be used to interpret the various material and sample properties.

Referring now to FIG. 7, a flowchart of an exemplary method 700 for operating a tissue imaging system, for instance the system 100 discussed above, is shown. At step 702, a tissue under test (TUT) sample to be imaged is received at a receptacle, the receptacle positioning the TUT sample between a resonator and an emitter. At step 704, the emitter directs near infrared light through the TUT sample and onto the resonator. At step 706, variations in absorption rates at the resonator are identified. At step 708, the identified variations in absorption rates are converted into outputs related to material properties of the TUT sample. Various additions and modifications to the above-described method may be contemplated.

As discussed above, the system 100 enables high-sensitivity imaging of human tissue. For example, it may be used for brain or breast tissue imaging to support the detection of cancerous cells. However, the system 100 is not limited to human tissue imaging. In various embodiments, it may be employed for the detection of pathogens in fluid-based samples, such as in identifying the presence of viruses, including but not limited to COVID-19. Additional contemplated implementations include the use of the system 100 in glucose monitoring sensors, exhaled-breath-based medical diagnostic systems, detection of specific gases in environmental or industrial settings (i.e., gas sensors), analysis of water quality or contamination (i.e., water purity sensors), antibody detection applications, quality control and industrial inspection systems, and sensors for detecting elements in wastewater streams. These examples are not limiting, and yet further use cases may be envisioned and implemented based on the core principles disclosed herein.

Referring now to FIGS. 8-16, in accordance with another embodiment of the present disclosure, there is provided a high-performance millimeter-wave (mm-wave) variable attenuator designed to operate across the full 75-110 GHz frequency band. Unlike conventional attenuators used solely for signal regulation, this variable attenuator is configured to act as a sensing mechanism, wherein attenuation levels are modulated in response to Near Infrared (NIR) illumination. The system leverages NIR-sensitive materials whose electromagnetic properties—such as permittivity or conductivity—change with incident light, resulting in measurable variations in transmission characteristics. As such, the attenuator becomes a functional sensor that detects changes in environmental or sample conditions based on light-induced responses. This sensing mechanism can be integrated into the above-described tissue imaging system 100, enabling detection of material properties through modulation of the microwave or millimeter-wave signal. While the 75-110 GHz frequency band is exemplified, other operational bands may be employed depending on application needs. In select embodiments, the structure includes a dielectric waveguide or image guide composed of NIR-sensitive materials, allowing precise correlation between NIR intensity and electromagnetic wave attenuation—forming the basis for a new class of photonic-interactive mm-wave sensors.

Various prior disclosures have investigated the applicability of Millimeter Wave (mmWave) and Terahertz (THz) technologies across a range of advanced domains, including high-data-rate wireless communications, precision sensing and imaging, and radio astronomy, among others. These technologies have demonstrated significant potential for both scientific and commercial applications due to their ability to support ultra-wide bandwidths and highly directional signal propagation. For instance, mmWave and THz technologies are explored in I. Dan et al., “A 300-GHz wireless link employing a photonic transmitter and an active electronic receiver with a transmission bandwidth of 54 GHZ,” IEEE Transactions on Terahertz Science and Technology, vol. 10, no. 3, pp. 271-281, May 2020; T. S. Rappaport et al., “Wireless communications and applications above 100 GHz: Opportunities and challenges for 6G and beyond,” IEEE Access, vol. 7, pp. 78729-78757, 2019; Michael S. Shur, “Terahertz Plasmonic Technology,” IEEE Sensors Journal, vol. 21, no. 11, pp. 12752, Jun. 1, 2021; and A.-A. A. Boulogeourgeos et al., “Terahertz Technologies to Deliver Optical Network Quality of Experience in Wireless Systems Beyond 5G,” IEEE Communications Magazine, vol. 56, no. 6, June 2018, pp. 144-151.

Additional advancements in the mmWave and THz fields have concentrated on the development of both signal generation and detection mechanisms, as well as the creation of functional components essential for enabling sensing and imaging applications. For example, recent efforts have explored the integration of these technologies into healthcare diagnostics and material testing. Notable contributions include Mavis Gezimati and Ghanshyam Singh's work, “Terahertz Imaging and Sensing for Healthcare: Current Status and Future Perspectives,” IEEE Access, vol. 11, 2023, which provides an in-depth analysis of the role of THz systems in biomedical imaging. Similarly, Walter Nsengiyumva et al., in “Sensing and Nondestructive Testing Applications of Terahertz Spectroscopy and Imaging Systems: State-of-the-Art and State-of-the-Practice,” IEEE Transactions on Instrumentation and Measurement, vol. 72, 2023, offer a comprehensive overview of THz spectroscopy and imaging systems applied to material inspection and nondestructive testing.

The development of low-cost, low-loss integrated circuit technology based on the THz platform is pivotal to realizing the full potential of emerging applications in this frequency range. Among critical components in mmWave-THz systems, the high-resolution variable attenuator plays a key role in enabling electronically controlled, high-performance systems operating in the THz domain. These attenuators are essential for tasks such as precise power adjustment in THz test equipment, source and link characterization, and sidelobe level control in antenna arrays. Design priorities for THz attenuators include compact form factor, cost-effectiveness, low insertion loss, and minimal power consumption. Important specifications comprise attenuation range, step resolution, bandwidth, and amplitude and phase accuracy. Nevertheless, planar transmission line-based attenuators often face significant transmission losses at higher frequencies. One strategy to mitigate these losses involves integrating passive components with on-chip active devices, as demonstrated in Mohamed Hussein Eissa and Gerhard Kahmen's work, “A 200-260-GHz Voltage-Controlled Distributed Attenuator in 130-nm BiCMOS: C Technology,” IEEE Microwave and Wireless Technology Letters, vol. 33, no. 12, December 2023, and in the DC-50 GHz CMOS design presented by Peng Gu, Dixian Zhao, and Xiaohu You, “A DC-50 GHZ CMOS Switched-Type Attenuator With Capacitive Compensation Technique,” IEEE Transactions on Circuits and Systems—I: Regular Papers, vol. 67, no. 10, October 2020.

A recent advancement in the field introduced a novel waveguide-based THz variable attenuator integrated with a piezoelectric motor, as described by Subash Khanal, Sofia Rahiminejad, Choonsup Lee, Jacob Willem Kooi, Robert Lin, and Goutam Chattopadhyay in “A Waveguide-Based Variable Attenuator for Terahertz Applications,” IEEE Transactions on Terahertz Science and Technology, vol. 14, no. 2, March 2024. This design showcases excellent transmission performance and desirable attenuation characteristics. However, its structural complexity poses limitations for integration into large-scale THz phased array systems, potentially restricting its broader applicability in scalable, high-frequency platforms.

Signal amplitude control—irrespective of associated phase changes—is a critical requirement in phased array systems. To address this, a variety of THz attenuator designs have been proposed in recent literature. While waveguide-based commercial variable attenuators are available at lower THz frequencies, they exhibit significant limitations in terms of versatility. Specifically, their bulky form factor, elevated power consumption, and integration complexity often make them unsuitable for next-generation, compact, and scalable applications. Meanwhile, recent efforts have also focused on analog component designs for millimeter-wave frequency bands, such as the work by B. Suh and B.-W. Min, “A 20-36-GHz voltage-controlled analog distributed attenuator with a wide attenuation range and low phase imbalance,” IEEE Transactions on Microwave Theory and Techniques, vol. 69, no. 5, pp. 2485-2493 May 2021, and I. Song, M.-K. Cho, and J. D. Cressler, “Design and analysis of a low loss, wideband digital step attenuator with minimized amplitude and phase variations,” IEEE Journal of Solid-State Circuits, vol. 53, no. 8, pp. 2202-2213 August 2018.

Numerous planar THz transmission lines and passive elements have also been introduced to support the development of low-loss, compact THz systems. Notable examples include the silicon-on-glass dielectric waveguide platform for millimeter-wave integrated circuits, as described in N. Ranjkesh et al., “Silicon-on-Glass Dielectric Waveguide—Part I: For Millimeter-Wave Integrated Circuits,” IEEE Transactions on Terahertz Science and Technology, vol. 5, no. 2, March 2015. Further advancements in this area are demonstrated in the development of a 1.1 THz U-shaped silicon-on-glass (U-SOG) waveguide platform for high-density THz integrated circuits, discussed in N. Ranjkesh et al., “1.1 THz U-Silicon-On-Glass (U-SOG) Waveguide: A Low-Loss Platform for THz High-Density Integrated Circuits,” IEEE Transactions on Terahertz Science and Technology, vol. 8, no. 6, November 2018. Additional progress is illustrated in hybrid metallo-dielectric waveguide architectures proposed by C.-M. Liu, L.-P. Carignan, and K. Wu in IEEE Microwave and Wireless Technology Letters, as well as in the fabrication of 3D-printed 1.1 THz waveguides, demonstrated by W. J. Otter et al. in “3D Printed 1.1 THz Waveguides,” Electronics Letters, vol. 53, no. 7, pp. 471-473, March 2017.

These high-frequency waveguides demonstrate low propagation losses in the THz range and enable wideband, single-mode operation, which is essential for high-performance signal transmission. Among various material candidates, alumina, quartz, and silicon (Si) have emerged as leading dielectric waveguide substrates in the millimeter-wave and THz frequency domains due to their ability to effectively minimize attenuation during electromagnetic wave propagation. Notably, silicon distinguishes itself through its exceptional optical transparency and low dispersion across the mmWave to THz spectral range, making it an optimal choice for dielectric waveguide construction in advanced sensing and imaging applications.

The selection of a Near Infrared (NIR)-sensitive material as the foundation for a THz dielectric waveguide enables the realization of low-cost and precisely controllable power modulation devices. However, such materials must meet specific criteria. In particular, the energy of NIR photons must correspond closely to the band gap energy of the silicon (Si) material used. In accordance with the present disclosure, an exemplary variable attenuator is constructed using doped silicon with high resistivity (approximately 10 kΩ·cm) and a band gap energy (Eg) of 1.12 eV. Exposure to modulated NIR radiation induces changes in the electrical conductivity of the doped Si, thereby enabling dynamic and accurate control of signal attenuation in the THz frequency domain.

The exemplary attenuator, discussed in further detail below, is capable of delivering a maximum attenuation range of 50 dB, with fine control steps as small as 0.1 dB, by utilizing doped silicon with a resistivity exceeding 10 kΩ·cm. The silicon image waveguide is integrated with WR10 rectangular metallic waveguides at both its input and output ends to ensure efficient coupling. The waveguide dimensions are designed to support single-mode Ey11 operation. In a representative implementation, the silicon waveguide is illuminated using three Near Infrared (NIR) light-emitting diodes (LEDs), collectively emitting 21 mW of optical power at a wavelength of 870 nm.

The interaction between the NIR radiation and the silicon waveguide induces absorption within the Si material, modifying its conductivity and thereby attenuating the propagating THz signal. This attenuation is achieved with minimal phase distortion—for example, attenuation levels of up to 20 dB can be realized with negligible phase impact. The structure features a compact footprint, low power requirements, and is particularly well-suited for low-THz wireless communication systems, phased array applications, and high-resolution sensing and imaging. Notably, the design offers a 50 dB dynamic range, enabling precise and tunable signal control for sensitive sensing and imaging applications.

Referring now to FIG. 8, a perspective view of an exemplary variable attenuator 800 is shown. The depicted attenuator 800 includes a metal fixture 802 and a rectangular waveguide 804 fabricated from high-resistivity silicon (HR-Si), which serves as the primary medium for guiding electromagnetic waves as an image guide. While this configuration is illustrated using HR-Si in a rectangular form, alternative shapes and materials may be contemplated. High-resistivity silicon is known for offering minimal electromagnetic losses across a wide frequency spectrum, spanning from microwave frequencies up to 10 THz, as demonstrated in J. Krupka et al., “Dielectric properties of semi-insulating silicon at microwave frequencies,” Applied Physics Letters, vol. 107, no. 8, August 2015, Art. No. 082105. Both doped and intrinsic forms of HR-Si possess energy gaps below 1.12 eV, where the dominant mechanism for signal attenuation is linked to conductivity resulting from free charge carriers.

Referring now to FIG. 9, a perspective cross-sectional view of a metal substrate 902 and a silicon image waveguide 904 for a variable attenuator 900 is shown. The conductivity of the silicon (Si) waveguide 904 can be precisely modulated using Near-Infrared (NIR) illumination. In the illustrated embodiment, the silicon waveguide 904, which features a sharpened end geometry, is integrated with standard WR-10 rectangular waveguides through metallic fixtures at both the input and output interfaces. This structural configuration allows seamless coupling of THz signals into and out of the silicon-based image guide.

Modal analysis of the silicon waveguide 904 can provide insights, for instance, into cut-off frequencies, propagation characteristics, losses, and field confinement, among other possible parameters. In the shown case, and based on the Si dimensions, either the E_{y}¹¹ or the Eₓ¹¹ mode will be dominant. When a width a of the waveguide 904 is greater than a height h of the waveguide 904, Eₓ¹¹ will be the dominant mode, whereas when a is less h, E_{y}¹¹ will be the dominant mode. Modal analysis of the Si image waveguide dimensional sizes is performed with High-Frequency Structure Simulator (HFSS) software to support the dominant E_{y}¹¹ mode from 75 up to 110 GHz. Exemplary dimensional sizes of Si are a=0.5 mm, h=0.7 mm, although other dimensions may be contemplated. In the simulation parameters of this design, High-Resistivity (HR) Si with a resistivity of >10 kΩ·cm was used. HR-Si with different values of resistivity has been characterized in the literature, for instance in M. N. Afsar and K. J. Button, “Millimeter-wave dielectric measurement of materials,” Proc. IEEE, vol. 73, no. 1, pp. 131-153, January 1985, in P. H. Bolivar et al., “Measurement of the dielectric constant and loss tangent of high dielectric-constant materials at terahertz frequencies,” IEEE Trans. Microwave. Theory Techn., vol. 51, no. 4, pp. 1062-166 April 2003, and in Jerzy Krupka, Jonathan Breeze, Anthony Centeno, Neil Alford, Thomas Claussen, and Leif Jensen “Measurements of Permittivity, Dielectric Loss Tangent, and Resistivity of Float-Zone Silicon at Microwave Frequencies” IEEE TRANSACTIONS ON MICROWAVE THEORY AND TECHNIQUES, VOL. 54, NO. 11, November 2006.

The effective dielectric loss tangent of the semiconductor was presented in K. S. Champlin and R. R. Krongard, “The measurement of conductivity and permittivity of semiconductor spheres by an extension of the cavity perturbation method,” IRE Trans. Microw. Theory Tech., vol. MTT-9, no. 11, pp. 545-551, November 1961, and can be expressed as:

$${tan}_{δ} = tan {δ}_{d} + σ / {ωε}_{0} {ε}_{r} \qquad ( 4 )$$

where ε₀ represents the permittivity of vacuum, εᵣ stands for the relative real permittivity of the semiconductor, ω denotes the angular frequency, σ indicates the conductivity of the semiconductor, and tan δ_{d} represents the dielectric loss tangent related to pure dielectric loss mechanisms.

For materials exhibiting such characteristics, their dielectric loss tangent values can significantly affect the second part of equation (4). Measurements have shown that the loss tangent value associated with pure dielectric losses for intrinsic Silicon is tan δ_{d}=1.2×100⁻⁵ at microwave frequencies, for instance in V. V. Parshin, R. Heidinger, B. A. Andreev, A. V. Gusev, and V. B. Shmagin, “Silicon as an advanced window material for high power gyrotrons,” Int J. Infr. Millim. Waves, vol. 16, no. 5, pp. 863-877, 1995.

In simulations where HR-Si conductivity changes under NIR illumination, a varied value for conductivity can be used since this conductivity is not precisely known. It is widely recognized that Light-sensitive intrinsic Si crystals possess high resistance (in dark conditions), primarily due to the lack of free electrons capable of movement. Most electrons are tightly bound within the crystal lattice, hindering their migration and resulting in high resistance. In simulations, we it can be assumed that HR-Si has a resistivity of 10 Kohm·cm, which can vary by up to 1 Kohm·cm under NIR illumination.

Various methods, for instance, doping, exist to increase the resistivity of Silicon. The introduction of dopants in both n- and p-type low-doped silicon crystals significantly enhances resistivity. When photons strike the Light-dependent Si, they excite electrons, promoting them from the valence band to the conduction band. This transition allows electrons to move freely, enabling the conduction of electricity. With increased light exposure to the High-resistive Silicon, more electrons are released, leading to heightened conductivity and reduced resistance. When light hits doped silicon, some of its valence electrons absorb energy and break their bonds. These electrons then transition to the conduction band for their lifetime, where they are not bound to any specific atom. Consequently, they can move freely from one location to another. These electrons, which can move without being tied to a particular atom, are referred to as free electrons. In the absence of illumination, it can be assumed that HR-Si conductivity is 10 kohm/cm which is equal 01=0.01 S/m. The permittivity of Si, εᵣ=11.7, is well-documented in literature, for instance in K. S. Champlin and R. R. Krongard, “The measurement of conductivity and permittivity of semiconductor spheres by an extension of the cavity perturbation method,” IRE Trans. Microw. Theory Tech., vol. MTT-9, no. 11, pp. 545-551, November 1961, in V. V. Parshin, R. Heidinger, B. A. Andreev, A. V. Gusev, and V. B. Shmagin, “Silicon as an advanced window material for high power gyrotrons,” Int J. Infr. Millim. Waves, vol. 16, no. 5, pp. 863-877, 1995, and in J. Krupka, P. Kaminski, and L. Jensen, “High Q-factor millimeter-wave silicon resonators,” IEEE Trans. Microw. Theory Techn., vol. 64, no. 12, pp. 4149-4154 December 2016.

Referring now to FIG. 10, a backside illumination model is shown for the variable attenuator 1000 having metal substrate 1002 and Si waveguide 1004. NIR illumination affects only a portion of the Si waveguide 1004, for instance an exemplary length of 10 mm. As such, the conductivity σ can be adjusted accordingly. For instance, In the S-parameters simulation for the structure depicted in FIG. 8, a value of εᵣ=11.7 can be used with varying conductivity values (o).

Referring now to FIG. 11, a graphical depiction 1100 of exemplary simulation results of the variable attenuator 800, 900, 1000 is shown. As shown, simulation results of scattering parameters for the attenuator 800, 900, 1000 are depicted with different conductivity numbers. In particular, the depicted simulation results of the variable attenuator S-parameters under varying intensities of irradiation were taken at σ=0.01 S/m, σ=0.012 S/m, σ=0.03 S/m, σ=0.7 S/m, and σ=1 S/m. As shown in these observations, attenuation can be changed in very small increments by varying the conductivity through illuminating with different intensities.

As shown in FIG. 11, within the exemplary frequency range of 75 to 110 GHz, the return loss remains better than 17 dB for various adjustable conductivities. The direct loss remains consistently around 1.8 dB across all frequency ranges. FIG. 12 shows a graphical depiction of exemplary modular phase shifting of the simulation results of FIGS. 11, at 1202, 1204, 1206, and 1208, in which an exemplary phase shift of approximately 2° is shown, with an attenuation of up to 14 dB.

Referring now to FIG. 13, an image 1300 of an exemplary variable attenuator 800, 900, 1000 in a fixture is shown. The depicted fixture is metallic, although other materials may be contemplated. Creating an optimal measurement setup with minimal errors is crucial for achieving accurate experimental results. The metallic fixture depicted in FIG. 13 serves as the foundation for the setup of the variable attenuator 800, 900, 1000. In an exemplary embodiment, the metallic fixture can be connected, via its input and output, to a 75-110 GHz VNA. The fixture encompasses several parameters, including dimensions, material conductivity, surface roughness, which collectively influence the measured accuracy of the system.

Precise error analysis of measurements allows for the evaluation of the system's performance within a real laboratory environment. Notably, errors can be categorized into fabrication and measurement uncertainties. To enhance experimental accuracy, various factors are considered, such as conductivity and surface roughness of the ground, fabrication technique, and implementation and loss reduction.

Referring to FIG. 14, an image 1400 of the variable attenuator in the fixture of FIG. 13 with an exemplary microwave absorber sheet is shown. As discussed above, the surface roughness of the ground can impact the experimental accuracy of the system described herein. While the dominant mode remains highly confined within the image guide, imperfectly conductive ground can increase the attenuation constant. Indeed, the metal surface roughness of the fixture can enable undesirable modes or introduce additional losses, such as radiation. To counteract these undesired modes, a microwave absorber sheets, for instance with a thickness of 1 mm, can be strategically positioned near the silicon (Si) image guide. This can suppress unwanted modes such as Eₓ¹¹ and high-order modes, thereby reducing measurement errors within the 75-110 GHz frequency range.

The fabrication technique of the disclosed system can also affect the experimental accuracy. The silicon image guide can, for instance, be fabricated using laser machining, offering advantages over traditional techniques like Deep Reactive Ion Etching (DRIE). For instance, laser machining is mask-free, chemical-free, rapid, and cost-effective, with precision down to a few microns. Notably, the tapered transition of the Si waveguide significantly impacts the direct loss of the modulator being prepared. Sharper tips result in lower direct losses. However, as achieving an extremely sharp tip via laser machining can be impractical, the tip can be manually sharpened, for instance, using a dicing machine. Subsequently, the Si waveguide is treated with a 10% solvent HF solution, for instance for five minutes, to reduce surface damage after dicing.

Implementation and loss reduction can also affect the experimental accuracy. Referring to FIG. 15, an image 1500 of an exemplary measurement set up for the variable attenuator 800, 900, 1000 is shown. Epoxy glue can be used to affix the Si waveguide onto a metallic surface, ensuring minimal losses. On the backside of the modulator fixture, a series connection of three LEDs can be used. For instance, Thorlabs LEDs emitting at 870 nm wavelength with a power of seven milliwatts can be used, among others. Exemplary measured results of the relationship between attenuation and near-infrared (NIR) illumination intensity changes are shown in FIG. 15. The illumination intensity of the LED was adjusted by applying varying volts. Additionally, FIG. 15 depicts the variation in phase with attenuation. Notably, it is observed that the phase undergoes exemplary evanescent changes up to −20 dB attenuation cases. Results of this phase change measurement with varying LED illumination are shown in FIG. 16, which is a graphical depiction of exemplary measurement results from the measurement set up of FIG. 15.

According to embodiments of the present disclosure, there is provided a system and method for high-sensitivity tissue imaging. In a particular embodiment, a high-quality Whispering Gallery Mode (WGM) silicon-based resonator serves as the core sensing element of the imaging system. To enable precise imaging, the tissue under test (TUT) specimen or sample is positioned atop the resonator, minimizing electromagnetic disturbance. Near-infrared (NIR) light is directed through the TUT sample onto the WGM resonator so that variations in Silicon absorption rates are exploited. Indeed, these variations induce changes in the transmission coefficient (S21) magnitude and/or phase, which are harnessed to conclude the sample's material properties. Notably, the high sensitivity of the WGM transmission coefficient, further amplified through precise Silicon doping, is harnessed to significantly improve tissue imaging quality.

There is also provided a low THz NIR attenuator, utilizing a Si image waveguide for transmission. Operating within an exemplary 75-110 GHz frequency range, this attenuator offers remarkable precision with an attenuation accuracy of 0.1 dB and a wide attenuation range of up to −50 dB. Notably, up to 20 dB of attenuation induces minimal phase alteration. Furthermore, its energy-efficient design consumes a mere 21 mV of power. These exceptional characteristics make it well-suited for integration into low THz antenna array systems, promising enhanced performance and versatility.

According to the present disclosure, there are also provided systems for molecular biomarker testing through immunoassays. Immunoassays are bioanalytical methods that exploit the specificity of antigen-antibody interactions, as noted in Arun Manickam, Rituraj Singh, Mark W. McDermott, Nicholas Wood, Sara Bolouki, Pejman Naraghi-Arani, Kirsten A. Johnson, Robert G. Kuimelis, Gary Schoolnik, and Arjang Hassibi “A Fully Integrated CMOS Fluorescence Biochip for DNA and RNA Testing” IEEE Journal of Solid-State Circuits, Vol. 52, NO. 11, 2017 pp 2857-2870 and in Marcin Kowalski, Mateusz Brodowski, Karolina Dzia bowska, Marta Skwarecka, Mateusz Ficek, Dawid Nidzworski, and Robert Bogdanowicz “Electrochemical Detection of Plant Pathogens Using Boron-Doped Carbon Nanowalls Immunosensor” IEEE Sensor Journal, Vol. 22, No. 8, 2022 pp 7562-7571. Antibodies, generated by the immune system in response to foreign molecules, bind to specific antigens with high affinity, forming antibody-antigen complexes. This interaction underpins the selectivity and sensitivity of immunoassays, which can be tailored to detect a wide range of molecular targets.

Immunoassay detection methods are typically classified into two primary categories: labeled techniques and label-free techniques. Labeled immunoassays employ detectable tags—such as enzymes, fluorophores, radioisotopes, or chemiluminescent molecules—attached to antibodies or antigens to facilitate detection, as noted in Hung-Wei Wu “Label-Free and Antibody-Free Wideband Microwave Biosensor for Identifying the Cancer Cells” EEE Transactions On Microwavw Theory And Techniques, Vol. 64, NO. 3, 2016 pp 982-990, in Muhammadeziz Tursunniyaz and Joseph Andrews “Printed Capacitive Immunoassay for Detecting SARS-COV-2 Viral Particles” IEEE Sensor Journal, Vol. 23, No. 20, 2023 pp 23975-23979, and in Marcin Kowalski, Mateusz Brodowski, Karolina Dzia bowska, Marta Skwarecka, Mateusz Ficek, Dawid Nidzworski, and Robert Bogdanowicz “Electrochemical Detection of Plant Pathogens Using Boron-Doped Carbon Nanowalls Immunosensor” IEEE Sensor Journal Vol 22 2022 pp 7562-7571. In contrast, label-free immunoassays directly measure antigen-antibody interactions via physical or chemical transduction methods, eliminating the need for tags. Label-free techniques are particularly advantageous for analyzing binding kinetics, though they depend on the development of highly specific antibodies. Advances in nanotechnology, microfluidics, and biosensor design have significantly improved the sensitivity and versatility of label-free immunoassay platforms, as noted in Hooriyeh Sadat Nourbakhsh, Nahid Raoufi, Kobra Omidfar, and Mehdi Ardjmand “A Sensitive Surface-Plasmon-Resonance (SPR)-Immunosensor Based on Multilayers of Graphene Oxide and Gold Nanoparticles for Epidermal Growth Factor Receptor Detection” IEEE Sensor Journal Vol 23 N0 1 2023 pp 300-307. Among these, surface Plasmon resonance (SPR) has emerged as a powerful tool for real-time analysis of binding kinetics. SPR detects changes in the refractive index near the sensor surface that occur during antigen-antibody binding (see FIG. 1). These changes are directly related to the antigen concentration, enabling the analysis of binding affinity and specificity without the need for labeling, as noted in Hsueh-Tao Chou, Yong-Sen Liao, Tien-Ming Wu, Shih-Han Wang, Kuan-Hua Chiang, and Wei-Chao Su “Development of Localized Surface Plasmon Resonance-Based Optical Fiber Biosensor for Immunoassay Using Gold Nanoparticles and Graphene Oxide Nanocomposite Film” IEEE Sensor Journal Vol 22 No 7 2022 pp 6593-6600.

Different biomarkers are known for assessing the presence of various substances in the bloodstream. For instance, calcidiol, or 25-hydroxyvitamin D3, is recognized as a reliable biomarker for assessing vitamin D status in the bloodstream, critical for diagnosing conditions like osteoporosis, rickets, and fracture risk. Monitoring calcidiol levels may offer a comprehensive assessment of vitamin D sufficiency or deficiency.

Referring to FIG. 17, there is provided an imaging system 1700 configured for small-molecule detection, in accordance with another embodiment of the present disclosure. Illustratively, a tissue under test (TUT) sample 1702 to be imaged using system 1700 is positioned between a NIR LED emitter 1704 and a Dielectric Resonator (DR) 1706, illustratively a Whispering Gallery Mode (WGM) silicon resonator. Illustratively, the TUT sample 1702 is a thin antigen-antibody layer that includes binding antibodies 1702a and antigens 1702b bound thereto. The TUT sample 1702 is positioned on a receptacle 1708 configured for receiving the TUT sample 102. As will be discussed in further detail below, the system 1700 is configured for identifying variations in absorption rates at the DR 1706 and converting the identified variations in absorption rates into outputs related to the material properties of the TUT sample 1702, and in particular of the antigen-antibody binding interactions. Various analyzers, for instance a Vector Network Analyzer (VNA), may be provided for receiving and analyzing results from the DR 1706.

In use, light from the NIR LED emitter 1704 passes through the thin antigen-antibody layer (TUT sample 1702) before interacting with the NIR-sensitive WGM silicon resonator (DR 1706) coupled to a microstrip line 1712. As such, even slight changes in light intensity on the DR 1706's surface will result in substantial shifts in the absolute value of the S21 parameter at the resonant WGM frequency, which is near-critically coupled to the microstrip line. As such, a direct description of antigen-antibody binding interactions is achieved.

In the shown embodiment, but not necessarily the case in all embodiments, the dielectric resonator (DR) 1706 is a whispering gallery mode (WGM) silicon-based resonator. Other materials sensitive to infrared light may be contemplated as well. DR 1706 may thus be referred to as a WGM resonator 1706. It is understood that other resonator types may also be contemplated. The WGM resonator 1706 is illustratively coupled with microstrip line 1712. In operation, as the NIR radiation emitted by the emitter 1704 interacts with the TUT sample 1702 (e.g., through transmission or reflection), it also illuminates the WGM resonator 1706 coupled with the microstrip line 1712. Illustratively, the WGM resonator 1706 and microstrip line 1712 are disposed on a platform 1714 or other like supporting surface. Consequently, this interaction leads to noticeable changes in transmission characteristics, including shifts in the amplitude and phase of transmission parameter S21, which measures the transfer of the signal power from the input to the output. These variations in the S21 parameter, quantified in the millimeter-wave frequency domain, directly correlate with the transmission and scattering of NIR radiation through the TUT sample 1702. Analysis of these variations by the VNA (or other analyzer) thus provides information regarding the material properties of the TUT sample 1702. In other embodiments, variations of the S12 transmission coefficient parameter are used in addition to, or in lieu of, the S21 parameter.

Referring now to FIG. 18, there is shown a schematic illustration 1800 of the dielectric resonator (DR) 1706 for the tissue imaging system of FIG. 17, according to some embodiments. As shown, a resonator and transmission line are provided, with various incident and reflected powers a1, a2, b1, b2, t and k to and from ports of the transmission line. As detailed in A. Yariv “Universal relations for coupling of optical power between microresonators and dielectric waveguides” Electronics Letters Vol 36 No 4 2000 pp 321-322 and in Amnon Yariv “Critical Coupling and Its Control in Optical Waveguide-Ring Resonator Systems” IEEE Photonics Technology Letters Vol. 14, No. 4, 2002 pp 483-485, the typical model of a resonator coupled to a transmission line yields equation (5) for the transmitted power at the resonance frequency, assuming negligible reflection and minimal losses in the coupling region |t|²+|k|²=1. The parameter k denotes the coupling coefficient and t denotes the field amplitude transmission past the coupling region. Power normalization is used so that |a₁|²=1 represents the incident power, and |b₁|₂ represents the transmission power.

$${\vert {b}_{1} \vert}^{2} = \frac{{( α - \vert t \vert )}^{2}}{{( 1 - α \vert t \vert )}^{2}} . \qquad ( 5 )$$

The transmitted power, |b|², becomes zero at a coupling value of α=|t|, a condition known as critical coupling. For high-Q resonators, where α is close to unity, even small variations in a for a given t can significantly affect the transmitted power |b|², allowing it to range from zero to unity. This power can be modulated by adjusting α, t, or both. The coupling, which is determined by the geometry of the resonator and the transmission line, is generally challenging to modify. However, the loss factor α can be altered through near-infrared (NIR) illumination when the resonator is a whispering gallery mode (WGM) silicon resonator. For example, high-resistivity silicon WGM resonators have been employed in applications such as tissue imaging and sugar detection, as noted in Amir Raeesi, Ala Eldin Omer, Afsaneh Hojjati-Firoozabadi, Aidin Taeb, Suren Gigoyan, and Safieddin Safavi-Naeini “Near Infrared-Controlled Whispering Gallery Mode Resonator Senso” IEEE Sensors Letters Vol 4 No 7 2020, and in Suren Gigoyan, Naimeh Ghafarian, Aidin Taeb, Mohammad-Reza Nezhad-Ahmadi, and Slim Boumaiza, “Tissue Imaging Technique Using Near-Infrared Illumination of Whispering Gallery Mode Silicon-Based Resonator” IEEE Microwave And Wireless Technology Letters Vol 34 Issue 10 pp 1210-1213.

Referring now to FIG. 19, there is shown an image 1900 of an exemplary dielectric resonator for the system 1700 of FIG. 17. As discussed above, the resonator 1906 and microstrip line 1712 are disposed on a platform 1914 or other like supporting surface. Illustratively, the platform 1914 is mounted to a base or supporting surface via clamps 1916. The depicted resonator 1906 is a WGM resonator fabricated from commercially available intrinsic silicon with a resistivity greater than ρ>50 kQ·cm, designed to sense NIR wavelengths at 870 nm and produced using laser machining. Other resonator types and fabrication means are contemplated. The microstrip line 1712 was fabricated on a Rogers substrate with dielectric properties of ε=6.15 and tan δ=0.0038. Other resonator and microstrip line types and fabrication means are contemplated.

Table II below details exemplary design parameters for the setup of FIG. 19, where t represents the substrate thickness, W is the width of the guiding strip, and L is the length. Additionally, Table II includes parameters for the fabricated sensor. It is understood that the following parameters are exemplary, and other parameters are contemplated.

TABLE II
Design Parameters of the Prepared Sensor

| Diameter | Thickness | Length of | Thickness of | With of guiding |
|---|---|---|---|---|
| WGM D | WGM T | the sensor L | substrate t | strip W |
| (mm) | (mm) | (mm) | (mm) | (mm) |
| D = 5.07 | T = 2.6 | L = 50 | t = 0.2 | W = 0.28 |

Typically, WGM resonators operating in low-order modes exhibit a low Q-factor due to radiation losses. According to present disclosure, to increase sensitivity, the system targets a high Q-factor in the WGH₉₀₀ mode, which is coupled with a microstrip line.

Referring now to FIG. 20, there is shown a graphical depiction 2000 of exemplary transmission coefficient parameters for the dielectric resonator of FIGS. 18-19. These baseline parameters are provided as a baseline when contrasted with the same measurements taken when a TUT sample is analyzed, as will be discussed in further detail below.

Referring now to FIG. 21, an image 2100 of an exemplary setup for the tissue imaging system 1700 of FIG. 17 is shown. As exemplary use case of the system will now be described. NIR light at 870 nm from an LED passes through a Plexiglas container to the lower surface, where an antibody-antigen thin layer is bound. This NIR light interacts with the thin antibody-antigen layer, causing evanescent changes in intensity that illuminate the whispering-gallery mode (WGM) silicon resonator. These evanescent changes in NIR intensity are related to the antibody-antigen coupling. Indeed, even small changes in NIR illumination lead to shifts in the loss tangent (tan δ) of the resonator, which in turn cause significant changes in the S21 parameter at resonant frequencies. To enhance sensitivity, the LED WGM Si resonator may be first illuminated through an empty plexiglass container, with the coupling subsequently adjusted to approach critical coupling. During the measurement, with an LED intensity of 0.5 mV, the resonator may achieve a critically coupled state, exhibiting up to 47 dB. Other setups for the system may be contemplated.

Referring now to FIG. 22, there is shown a graphical depiction 2200 of an experimental phase for the dielectric resonator of FIGS. 18-19 in response to changes in Near Infrared Light. FIG. 22 graphically depicts the transmission coefficient S21 through the resonator based on the frequency of emitted light in three specific test cases: an empty vessel (shown in black), a vessel having an antibody coating (shown in red) and a vessel having an antibody-antigen coating (shown in blue). The exemplary vessel for this testing is a plexiglass container with its bottom coated based on the specific case, although this is merely an example.

As shown in FIG. 22, there is a marked difference in the S21 transmission coefficient between the vessel with an antibody coating versus the vessel with an antigen-antibody coating. Indeed, a 10 db difference in amplitude in the S21 transmission coefficient is noted due to the presence of an antigen. The system as herein disclosed is therefore adapted to detect the presence of antigens based on the transmission coefficient detected upon shining light through the TUT sample and observing the results at the resonator. 2202 shows an example with an empty vessel. 2204 shows an example S21 with an antibody coating. 2206 shows an example S21 with an antibody-antigen with significant differentiation between 2202 and 2204.

FIG. 23A is an image 2300A showing the Si ring resonator, according to some embodiments.

FIG. 23B is an image 2300B showing a sample under test, as well as a coupled indicator LED, according to some embodiments.

According to the present disclosure, a novel immunoassay technique is provided, leveraging the high sensitivity of a Whispering Gallery Mode (WGM) silicon resonator excited by near-infrared (NIR) light. The WGM resonator, characterized by a high-quality factor (Q), functions as the primary sensing element for detecting specific antigen-antibody interactions within the NIR spectrum. In an exemplary embodiment, antigen-antibody complexes are immobilized at the base of a transparent Plexiglas container. NIR light, emitted from an LED, traverses the sample and interacts with the silicon WGM resonator, resulting in modifications to the Q factor and corresponding variations in the transmission coefficient (S21) at the resonant frequency. These S21 shifts are directly correlated to the presence and state of antigen-antibody binding, thereby enabling highly sensitive biomolecular detection. The described technique offers a simple, cost-effective, and portable diagnostic solution, suitable for use in various settings, including virus detection and blood analysis for cancer diagnostics.

Various modifications and enhancements to the above-described systems are contemplated. While the disclosed embodiments utilize near-infrared (NIR) light as the excitation source, other frequencies across the electromagnetic spectrum are also contemplated, including ultraviolet (UV), visible, terahertz (THz), and millimeter-wave light. In such cases, the resonator may be appropriately configured or selected to be sensitive to the corresponding wavelength.

Moreover, while the current resonator configuration is designed to operate based on a single resonance, alternative embodiments may employ multi-resonance or multi-frequency sensing architectures to enhance performance and broaden sensing capabilities. Although silicon is disclosed as the exemplary NIR-sensitive material, other resonator materials may be employed, including ferrite-based structures or various other dielectric or semiconducting compounds.

In addition to sensing via shifts in the transmission coefficient (S21), other detection mechanisms are also contemplated. These include monitoring phase variations of the transmission coefficient as supplementary data points and analyzing resonance frequency shifts corresponding to different sample states. In embodiments where ferrite materials are utilized, the inherent non-reciprocal behavior introduces distinct resonant frequencies for S21 and S12, which can both be used as independent sensing modalities.

Lastly, although the above embodiments describe the use of a flat substrate, other substrate configurations may be contemplated. For instance, the substrate may be flexible and conformal, enabling integration with non-planar surfaces or wearable sensing platforms.

Various implementations and use cases of the above-described technology are contemplated. For example, the disclosed sensing platform, which detects measurable amplitude changes in response to specific interactions or sample conditions, can be employed for rapid testing of viruses, bacteria, and other pathogens. Such implementations may be suited for both laboratory environments and at-home diagnostic use.

Beyond pathogen detection, the sensing system may be adapted and calibrated for alternative biomedical or environmental applications, such as:
Blood glucose monitoring
Cancer detection
Food safety inspections

In these contexts, the system can be configured to target specific biomarkers or contaminants, depending on the application. Furthermore, a compact, low-power portable sensor is contemplated, incorporating a transmit-and-receive chip capable of executing the above-described functionality. This enables integration into mobile, wearable, or remote diagnostic platforms, supporting real-time, high-sensitivity detection in diverse settings.

Various embodiments of tissue imaging systems and methods are disclosed herein. It is understood that the features and functionalities described in connection with one embodiment may be applicable to and interchangeable with those of other embodiments presented throughout this disclosure. Such cross-applicability enables modular implementation and flexible system integration across a wide range of imaging and sensing configurations.

Applicant notes that the embodiments and examples described herein are illustrative and non-limiting. Practical implementations may incorporate some or all of the disclosed features, and the inclusion of specific functionalities should not be interpreted as reflecting current or future product plans. Applicant engages in both foundational and applied research, and in certain instances, the described features are being developed or explored on an experimental or exploratory basis.

The terms “connected” or “coupled to” as used herein are intended to encompass both direct coupling—where two elements are in physical contact—and indirect coupling, wherein one or more intermediate elements may be positioned between the coupled elements.

Although the embodiments have been described in detail, it should be understood that various modifications, substitutions, and alterations may be made without departing from the scope of the present disclosure. Moreover, the scope of the present application is not intended to be limited to the specific embodiments of processes, machines, manufactures, compositions of matter, means, methods, or steps described herein.

As will be readily appreciated by those of ordinary skill in the art in view of the present disclosure, processes, machines, manufactures, compositions of matter, means, methods, or steps—whether presently existing or subsequently developed—that perform substantially the same function or achieve substantially the same result as the embodiments described herein may be utilized. Accordingly, such alternatives and equivalents are intended to be encompassed within the scope of the present disclosure.

It will be understood that the foregoing examples and illustrations are provided for purposes of explanation and are not intended to be limiting. Rather, they are exemplary in nature, and variations and modifications may be made without departing from the scope of the invention as defined by the appended claims.

We claim:

1. A diagnostic imaging and biosensing system comprising:
a receptacle configured to receive a tissue-under-test (TUT) sample or biological specimen;
a sensing structure configured to detect changes in optical or electromagnetic properties of the TUT sample in response to near-infrared (NIR) illumination, the sensing structure selected from the group consisting of:
(i) a near-infrared sensitive resonator coupled to a microstrip transmission line or a dielectric waveguide, and
(ii) a non-resonant near-infrared sensitive dielectric waveguide;
an NIR light emitter configured to direct NIR illumination through or onto the TUT sample and toward the sensing structure, wherein the system is operable in either:
(i) transmission mode, where light passes through the sample before interacting with the sensing structure, or
(ii) reflection mode, where reflected light from the sample is coupled into the sensing structure;
a signal detection and processing unit configured to:
acquire measurement data based on the interaction of the NIR signal with the sensing structure;
analyze one or more scattering parameters (S11, S21, S12, S22) and identify changes in at least one of:
(a) magnitude,
(b) phase, or
(c) resonance frequency, where applicable; and
generate output data indicative of one or more physical, chemical, or biochemical properties of the TUT sample, including antigen-antibody interactions or material composition.

2. The system of claim 1, wherein spatially resolved imaging includes:
(a) providing a two-dimensional translation stage for displacing the tissue-under-test (TUT) sample in controlled increments, such that changes in scattering parameters induced by NIR interaction with the sample are used to construct a two-dimensional image map of material properties;
or
(b) employing a two-dimensional array of sensing structures, each comprising a near-infrared (NIR)-sensitive resonator or dielectric waveguide coupled to a microstrip line or dielectric waveguide, the array being configured to simultaneously capture optical responses from distributed regions of the TUT sample for reconstruction of a spatial image without requiring mechanical movement.

3. The system of claim 2, wherein the two-dimensional image map is processed by a graphical display controller configured to render one or more visual interface elements that differentiate between tissue types based on variations in near-infrared absorption or scattering characteristics.

4. The system of claim 1, wherein the variations in absorption rates at the resonator are indicative of antigen-antibody interactions, the system being configured to detect one or more antigen-antibody binding events based on changes in at least one of the magnitude, phase, or resonance frequency of the resonator's electromagnetic response.

5. The system of claim 4, wherein the processor is configured to analyze the variations in absorption rates to determine spatial locations within the tissue under test (TUT) sample that correspond to regions containing antibody molecules alone or antibody-antigen complexes, thereby enabling biochemical mapping of the TUT sample.

6. The system of claim 1, wherein the sensing structure comprises one of:
(a) a whispering gallery mode (WGM) resonator composed of silicon, a near-infrared (NIR)-sensitive material, or a hybrid material exhibiting both NIR sensitivity and magnetic responsiveness; or
(b) a dielectric waveguide configured to be NIR-sensitive and to operate as a non-resonant sensing element;
wherein the system is further configured to operate in a reflection mode in which NIR light emitted by the emitter is reflected from the tissue under test (TUT) sample and coupled or recoupled into the sensing structure, and wherein the processor is configured to analyze variations in electromagnetic response, including at least one of magnitude, phase, or resonance frequency shift of one or more scattering parameters (S11, S21, S12, S22), to generate diagnostic or material property output data of the TUT sample.

7. The system of claim 6, wherein the receptacle is configured for single-sided access to the tissue under test (TUT) sample, enabling operation in a reflection-mode configuration in which near-infrared (NIR) light is directed toward the exposed surface of the TUT sample and a portion of the reflected signal is coupled into a sensing structure, the sensing structure comprising at least one of:
(a) a whispering gallery mode (WGM) resonator;
(b) a dielectric waveguide;
(c) a hybrid resonator comprising both magnetically sensitive (e.g., ferrite-based) and NIR-sensitive materials; or
(d) an array of such resonators or waveguides configured to enable spatially resolved imaging or biosensing.

8. The system of claim 1, wherein the microstrip line is configured with an adjustable geometry adapted to achieve near-critical coupling to the resonator at high quality factor (Q-factor) resonances, thereby enhancing the sensitivity of the system and improving the ability of the processor to detect induced changes in the transmission coefficient.

9. The system of claim 1, wherein the whispering gallery mode silicon-based resonator is doped with one or more Group III elements and further comprises a hybrid magnetic structure, enabling combined near-infrared and magnetic field sensitivity to enhance biosensing performance.

10. The system of claim 9, wherein the whispering gallery mode silicon-based resonator comprises aluminum-doped silicon and is configured to support a seventh-order whispering gallery mode resonance within microwave to terahertz frequency range.

11. A method for tissue imaging and biochemical sensing using near-infrared illumination and a whispering gallery mode silicon-based resonator, the method comprising:
receiving, at a receptacle, a tissue under test (TUT) or biological sample;
coupling a near-infrared (NIR)-sensitive resonant structure—comprising a whispering gallery mode (WGM) silicon-based resonator or a dielectric waveguide—to a microstrip line or dielectric transmission line;
directing, by an NIR light emitter, near-infrared illumination through or onto the TUT sample, wherein in a transmission mode the light passes through the sample onto the resonator, and in a reflection mode the light reflects off the sample and is re-coupled into the resonator;
detecting, in real time, variations in absorption rates or optical characteristics at the resonator or waveguide, wherein such variations induce changes in at least one of a magnitude, phase, or resonant frequency of one or more scattering parameters (S11, S21, S12, S22);
generating, by a processor, one or more output data structures representative of localized material or biochemical properties of the TUT sample;
correlating the detected variations with specific molecular interactions, including antigen-antibody binding events in immunoassay formats; and
rendering diagnostic results in a visual or digital form suitable for point-of-care, clinical, or field-deployable applications.

12. The method of claim 11, wherein the receptacle includes a two-dimensional translation stage configured to move the tissue under test (TUT) sample in controlled increments, and wherein the processor uses changes in the transmission coefficient, induced by the movement, to generate a localized mapping of different regions of the TUT sample, the mapping forming a two-dimensional image map incorporated into the one or more output data structures.

13. The method of claim 12, further comprising utilizing the 2-D image map as input to a graphical display controller configured to render one or more visual interface elements that distinguish between different tissue types and/or indicate regions of antigen-antibody binding activity, based on localized variations in absorption characteristics.

14. The method of claim 11, wherein the variations in the absorption rates at the resonator are indicative of antigen-antibody interactions, the method comprising detecting one or more antigen-antibody binding events based on changes in at least one of a magnitude, a phase, or a resonance frequency of a scattering parameter of the resonator.

15. The method of claim 14, wherein the processor is configured to analyze the variations in absorption rates to spatially resolve regions within the TUT sample containing unbound antibodies versus regions containing antibody-antigen complexes, thereby enabling molecular-level differentiation within the sample.

16. The method of claim 11, wherein the whispering gallery mode silicon-based resonator is configured in a ring-shaped geometry, and the method further comprises operating the system in a reflection-mode configuration by directing near infrared light onto a single accessible side of the TUT sample, receiving, at the resonator, a portion of the light reflected from the TUT sample, and processing characteristics of the reflected light using the processor to derive optical data indicative of induced changes in the transmission coefficient of the resonator.

17. The method of claim 16, wherein the receptacle is configured to provide single-sided access to the TUT sample, thereby enabling reflection-mode imaging by allowing near infrared light to be directed onto and reflected from a surface of the TUT sample toward a sensing structure comprising a whispering gallery mode (WGM) resonator or a near infrared-sensitive dielectric waveguide.

18. The method of claim 11, wherein the sensing structure comprises either a microstrip line or a dielectric waveguide, the sensing structure being configured with an adjustable geometry or refractive profile to enable near-critical coupling with a whispering gallery mode resonator or a non-resonant near-infrared-sensitive waveguide, thereby enhancing signal sensitivity and improving the processor's ability to detect induced changes in one or more scattering parameters, including transmission and reflection coefficients.

19. The method of claim 11, wherein the whispering gallery mode silicon-based resonator is doped with Group III elements and further incorporates hybrid magnetic characteristics configured to induce non-reciprocal propagation of electromagnetic waves, thereby enhancing sensitivity and selectivity in detecting variations in material properties of the tissue under test (TUT) sample.

20. A non-transitory computer-readable medium storing machine-interpretable instructions which, when executed by one or more processors, cause the processor(s) to perform a method for tissue imaging using near-infrared illumination of a whispering gallery mode (WGM) silicon-based resonator coupled with a microstrip line, the resonator being directed at a tissue under test (TUT) sample positioned on a receptacle, the method comprising:
identifying variations in absorption rates at the WGM resonator induced by the near-infrared illumination passing through or reflecting from the TUT sample; and
converting the identified variations in absorption rates into one or more output data structures representative of one or more material properties of the TUT sample;
wherein the variations in absorption rates at the WGM resonator cause changes in at least one of a magnitude and a phase of a transmission coefficient of the WGM resonator.
